SEQUENCE LISTING (21 sequences)

SEQ ID NO: 1 - DNA, 1416 bp, Reovirus
gctattggtcggatggatcctcgcctacgtgaagaagtagtacggctgataatcgcattaacgagtgataatggagcatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacgttagagcgcacggcggtcactagcgcgggagctcccctctcaatccgtaataaccgtatgaccatgggattaaatgatggactcacgttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggataggcgcaactgagcaaagttacgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 2 - DNA, 1331 bp, Reovirus
gctattcgctggtcagttatggctcgcgctgcgttcctattcaagactgttgggtttggtggtctgcaaaatgtgccaattaacgacgaactatcttcacatctactccgagctggtaattcaccatggcagttaacacagtttttagactggataagccttgggaggggtttagctacatcggctctcgttccgacggctgggtcaagatactatcaaatgagttgccttctaagtggcactctccagattccgttccgtcctaaccaccgatggggagacattaggttcttacgcttagtgtggtcagctcctactctcgatggattagtcgtagctccaccacaagttttggctcagcccgctttgcaagcacaggcagatcgagtgtacgactgcgatgattatccatttctagcgcgtgatccaagattcaaacatcgggtgtatcagcaattgagtgctgtaactctacttaacttgacaggttttggcccgatttcctacgttcgagtggatgaagatatgtggagtggagatgtgaaccagcttctcatgaactatttcgggcacacgtttgcagagattgcatacacattgtgtcaagcctcggctaataggccttgggaatatgacggtacatatgctaggatgactcagattgtgttatccttgttctggctatcgtatgtcggtgtaattcatcagcagaatacgtatcggacattctattttcagtgtaatcggcgaggtgacgccgctgaggtgtggattctttcttgttcgttgaaccattccgcacaaattagaccgggtaatcgtagcttattcgttatgccaactagcccagattggaacatggacgtcaatttgatcctgagttcaacgttgacggggtgtttgtgttcgggttcacagctgccactgattgacaataattcagtacctgcagtgtcgcgtaacatccatggctggactggtagagctggtaaccaattgcatgggttccaggtgagacgaatggtgactgaattttgtgacaggttgagacgcgatggtgtcatgacccaagctcagcagaatcaagttgaagcgttggcagatcagactcaacagtttaagagggacaagctcgaaacgtgggcgagagaagacgatcaatataatcaggctcatcccaactccacaatgttccgtacgaaaccatttacgaatgcgcaatggggacgaggtaatacgggggcgactagtgccgcgattgcagcccttatctgatcgtcttggagtgagggggtccccccacacacctcacgactgaccacacattcatc

SEQ ID NO: 3 - DNA, 1198 bp, Reovirus
gctaaagtcacgcctgtcgtcgtcactatggcttcctcactcagagctgcgatctccaagatcaagagggatgacgtcggtcagcaagtttgtcctaattatgtcatgctgcggtcctctgtcacaacaaaggtggtacgaaatgtggttgagtatcaaattcgtacgggcggattcttttcgtgcttagctatgctaaggccactccagtacgctaagcgtgagcgtttgcttggtcagaggaatctggaacgtatatcgactagggatatccttcagactcgtgatttacactcactatgtatgccaactcctgatgcgccaatgtctaatcatcaagcatccaccatgagagagctgatttgcagttacttcaaggtcgatcatgcggatgggttgaaatatatacccatggatgagagatactctccgtcatcacttgccagattgtttaccatgggcatggctgggctgcacattaccactgagccatcttataagcgtgttccgattatgcacttagctgcggacttggactgtatgacgctggctctaccttacatgattacgcttgatggtgatactgtggttcctgtcgctccaacactgtcagcggaacagcttctggacgacggactcaaaggattagcatgcatggatatctcctatggatgtgaggtggacgcgaatagccggccggctggtgatcagagtatggactcttcacgctgcatcaacgagttgtattgcgaggagacagcagaagccatctgtgtgcttaagacatgccttgtgttaaattgcatgcagtttaaacttgagatggatgacctagcacataacgctgctgagctggacaagatacagatgatgatacccttcagtgagcgtgtttttaggatggcctcgtcctttgcgactattgatgcccagtgttttaggttttgcgtgatgatgaaggataaaaatctgaaaatagatatgcgtgaaacgacgagactgtggactcgttcagcatcagatgattctgtggccacgtcatctttaagtatttccctggaccggggtcgatgggtggcggctgacgccagtgatgctagactgctggtttttccgattcgcgtgtaatgggtgagtgagctgatgtggtcgccaagacatgtgccggtgtcttggtggtgggtgacgcctaatcatc

SEQ ID NO: 4 - DNA, 1196 bp, Reovirus
gctatttttgcctcttcccagacgttgtcgcaatggaggtgtgcttgcccaacggtcatcaggtcgtggacttaattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaaccggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactgaagggagatgctcgtcaaacccaattctccaggacttttgattcgagatcgagtttggaatggggtgtgatggtttatgattactctgagctggatcatgatccatcgaagggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattatccagtgatgattggcgatccgatgattcttggctaaacacccccatcttcacagcgccgggcttgaccaacctggtgtgacgtgggacaggcttcattcatc

SEQ ID NO: 5 - DNA, 2304 bp, Reovirus
gctattcgcggtcatggcttacatcgcagttcctgcggtggtggattcacgttcgagtgaggctattggactgctagaatcgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcgaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttcctgtatgtattcttgaaatgttgggtattgagattgcggactattgcattcgtcaagaggatggaatgctcacagattggttcctacttttaaccatgctatctgatggcttgactgatagaaggacgcattgtcaatacttgattaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgttgaactcggatctcattccgcatgatgacaggcttggtcaattatttcaagcgtttttgcctctcgcaaaggacttattagctccaatggccagaaagttttatgataactcaatgagtgagggtagattgctaacattctctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 6 - DNA, 2204 bp, Reovirus
ggctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggacacggcaataatcaacacagacaattcatcaggcgccgtgccaagcgaatcagccttggtgccctacatcgatgagccgctggtagtggttacagagcatgctattaccaacttcaccaaagctgagatggcacttgaattcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacagcctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgcgccagttcccgcacccgtgtttgccataccacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgtattagatctagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 7 - DNA, 2241 bp, Reovirus
gctaaagtgaccgtggtcatggcttcattcaagggattctccgccaacactgttccagtttctaaggccaagcgtgacatatcatctcttgccgctactcctggacttcgttcacaatccttcactccgtctgtggatatgtctcaatcgcgtgaattcctcacaaaggcaattgagcaagggtccatgtctataccttatcagcatgtgaatgtaccgaaagttgatcgtaaagttgttagcctggtagtgcgacctttctcttcaggtgctttctctatctctggagtgatttcgccagcccatgcctatctactagagtgtctaccccagcttgagcaggcgatggcttttgttgcttcacctgagtctttccaggcttccgacgtcgcgaagcgctttgccataaagccaggtatgagcctccaggatgccatcactgcctttattaactttgtgtccgcgatgctgaaaatgacggtgactcgtcaaaactttgacgttattgtggctgagatcgagaggcttgcttcaaccagcgtgtccgtcaggactgaagaagcgaaggttgctgatgaggagctaatgctattcgggttagatcatagagggccacagcagctggatgtttctgacgctaaagggataatgaaggctgctgatattcagacaactcatgatgtccatttggcaccaggcgttggtaatattgatcctgaaatctataacgaggggcggttcatgttcatgcagcacaagccacttgcggcggatcaatcgtatttcaccttggagactgcggattatttcaagatttatccaacatacgatgaacatgatggcaggatggctgaccaaaagcagtcgggattgatactgtgtactaaggacgaggtattggctgagcaaactatatttaaactggacgcccctgatgacaagactgttcatctgttggatcgcgatgacgaccacgttgttgccagatttactaaggtatttatagaggacgtggctcccgggcatcatgctgctcaaagatcgggacaacgctctgtgcttgatgacctatatgcgaatacgcaagtgatttccattacttctgctgctttaaagtgggtggtcaagcacggcgtatctgatggaatcgtgaacaggaagaatgtcaaagtgtgtgttggttttgaccccctgtacaccttgtctacacataacggggtgtccttatgtgccctgctgatggacgaaaaactctctgtgctgaacagtgcgtgtcgtatgacgttacgctcactcatgaagaccggacgcgacgttgatgcacacagagcttttcagcgagtcctctctcaaggatacacatcgctaatgtgctactatcatccttcacggaagttggcatatggtgaggtgctctttctagaacgatccaatgacgtgacagatgggatcaagcttcagttggacgcatctagacagtgtcatgaatgtcctgtgttgcagcagaaagtggttgagttagagaaacagattattatgcagaagtcaatccagtcagaccctaccccagtggcgctgcaaccattgttgtctcagttgcgtgagttgtctagtgaagttactaggctacagatggagttgagtcgagctcagtccctgaatgctcagttggaggcggatgtcaagtcagctcaatcatgtagcttggatatgtatctgagacaccacacttgcattaatggtcatgctaaagaagatgaattgcttgacgctgtgcgtgtcgcgccggatgtgaggagagaaatcatggaaaagaggagtgaagtgagacaaggttggtgcgaacgtatttctaaggaagcagctgccaaatgtcaaactgttattgatgacctgactttgatgaatggaaagcaagcacaagagataacagaattacgtgattcggctgaaaaatatgagaaacagattgcagagctggtgagtaccatcacccaaaaccagataacgtatcagcaagagctacaagccttggtagcgaaaaatgtggaattggacgcgttgaatcagcgtcaggctaagtctttgcgtattactccctctcttctatcagccactcctatcgattcagttgatgatgttgctgacttaattgatttctctgttccaactgatgagttgtaaataatccgtgatgcagtgttgccctaatcccttaagccttcccgacccccattcatc

SEQ ID NO: 8 - DNA, 3854 bp, Reovirus
gctacacgttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggttttctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttatgcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcaaccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaagatgctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcacccgtattcgggaacgcagattatcgcaggtgtttcaatgaacttaaactatatcaaggttattatatggcacaattgcccaggaatcctaagaagtctggacgagcggcccctcgggaggtaagagaacaattcactcaggcattatccgactatctactgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagatattcgagcttttcaaagttattagaggcgtatctgctcgtgaaatggcgaatgtgcgaggcccgcgaaccgtcggtggatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgactctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 9 - DNA, 3916 bp, Reovirus
gctaaatggcgcgatggcgaacgtttggggggtgagacttgcagactcgttatcttcacccactattgagacacgaacgcgtcagtataccttacacgatctttgctcagacctagatgctaatccggggagggaaccgtggaaacctctgcgtaatcagcgtactaataatattgtggctgtgcaattattcagaccattgcagggtttagttttagatacccagctttatggatttccaggagcatttgatgactgggagcgattcatgagagagaagctgcgtgtgctaaagtatgaagtattgcgcatctatccaatcagcaactatagcaatgaacatgtcaacgtcttcgtggccaatgctttggtgggcgctttcctgtcgaatcaagctttctatgacctgctaccgttgttgataattaatgacactatgattggtgatctacttggcacgggggcatcgctatcacagttctttcaatctcatggagatgtgctggaagtcgcagctggtcgtaagtatctgcagatggaaaactactccaacgatgacgatgatcctccattatttgcgaaagacctgtcagattatgctaaagcattctacagtgacacatatgaagtgttggacaggttcttttggacgcatgactcttcagcgggggtcttagtgcattatgataagccaacgaatggtcatcactatctgctgggtactttgactcagatggtcagtgcacctccttatattattaacgctactgacgcaatgttgcttgaatcctgtctagaacagttctcagctaatgtgcgtgcgagacctgcgcaacccgttacacgcttagaccaatgctatcatttaagatggggagcacaatatgtaggagaagattcactgacatatcggttgggggtgttatccttgctggctaccaatggatatcaattagctagaccgattccaagacagttgacgaatcgatggttgtcgagctttgtgagtcaaattatgtctgacggcgtcaacgagactccactgtggccccaagaaaggtatgtgcagatcgcttatgattcaccatccgttgttgatggggctacgcaatatggctatgtcaggaagaatcaactcagactcggcatgagaatatcggcgctgcaatcgctgagtgatacgccctcgccggtacagtggcttccacaatacaccatcgaccaggcagcgatggacgaaggcgatctgatggttagtcggcttacgcaactcccgttacgtcctgattatggtaatatctgggtcggcgatgcgctatcctattatgtggactacaatcggagtcatcgagtcgtgctttcatcggaacttcctcagcttccggacacatattttgatggcgatgaacagtatgggcgcagcctgttctcactagctcgtaagattggtgaccgctcgttagtgaaagatacggctgtcttgaagcacgcttaccaagccatcgatccaaatactggtaaggagtatctgagatctcggcaatctgtcgcatattttggtgcatcagcgggtcattctggtgccgaccagccgttagtcatagagccctggattcaagggaaaatcagtggtgtgccgccaccctcctcagtgcgacagttcggctatgatgttgcccgtggcgcgatcgtcgatctggcgagaccatttccttctggagattatcaatttgtctattcggatgttgaccaggtggtcgatggccatgacgatctgagtatatcatctggactggtggagagccttttgtcttcatgcatgcacgccacagcacccgggggctcatttgttgttaagataaattttccgactagacccgtatggcactacatcgaacagaagatcttgcccaatattacgtcatacatgttgatcaagcctttcgtcaccaacaacgtcgaattgttcttcgtcgctttcggtgtgcatcaacactcatcacttacttggacatctggagtgtacttcttcttggtggaccatttttatcgttatgagactttatctacgatctcacgacaattgccgtcttttgggtatgttgatgatgggtcttccgtgactggtatcgagacaattagtattgagaaccctggcttctcgaatatgacccaggccgctcgcattggtatctcaggattgtgtgctaatgtaggtaacgcgcgtaagtccattgccatttacgaatctcatggggccagagtattaactatcacatcaaggagatctccggcatcagctagaagaaagtctaggttgcgatatttgccattaatagaccctaggtcgttagaggtacaggcgcgcactattctgccagctgatccagtgttatttgaaaacgtgagcggagcgtcaccccatgtttgtctgacaatgatgtacaacttcgaagtgtcgtcagcggtatatgatggagacgttgtgctagatcttgggacgggaccagaggctaaaatccttgaactgatacccgcaacctctccagtcacatgcgtggacatacggcctacagcgcagcctagtggatgttggaacgttcgtaccacgttccttgagttagattatttgagcgatggatggatcactggggtgcgtggggacatagttacttgtatgttatctttgggggccgctgccgctggaaaatcaatgacttttgacgctgcgtttcagcaattaatcaaagtattatccaagagtacggctaatgttgtgctggtgcaggttaactgccctacagacgtggtgaggagcattaagggctacctagagatagattcgactaacaagaggtataggttccccaaatttggtcgagacgagccgtactctgacatggatgcgctggagaaaatatgtcgtaccgcctggccaaactgctcaattacctgggttccattgtcatacgacttgcggtggactagactggcattattagagtccacgacattgagtagcgcgtcgattagaattgctgagctgatgtataaatacatgcctattatgaggattgatattcatggactacccatggaaaagcgaggtaacttcatagtggggcagaactgctcattagtaatccctggttttaatgcgcaggatgtctttaactgttatttcaattccgccctcgctttctcgactgaagatgtcaatgctgcgatgattccccaagtgtctgcgcagtttgatgcgactaagggtgagtggacgttggatatggtcttctccgacgcaggaatctataccatgcaggctctagtgggatctaatgctaatccagtctctttgggttcctttgtagttgattctccagatgtagatataactgacgcttggccagctcagttagactttacgatcgcgggaactgatgtcgatataacagttaatccttattaccgtctgatgacctttgtaaggatcgatggacagtggcagattgccaatccagacaaatttcaattcttttcgtcggcgtctgggacgttagtgatgaacgtcaaattagatatcgcagataaatatctactatactatatacgagatgtccagtctcgagatgttggcttttacattcagcatccacttcaacttttgaatacgatcacattgccaaccaacgaggacctttttctgagcgcacctgacatgcgagagtgggcagttaaggaaagcggtaacacgatatgtatactcaatagtcaagggtttgtgctacctcaagattgggatgtgttaacagataccataagttggtccccatcgatacccacatacattgtgccaccgggtgattataccttgactcctctgtaactcactgtccctcgtgagcgcgcctaattcatc

SEQ ID NO: 10 - DNA, 3901 bp, Reovirus
gctaatcgtcaggatgaagcggattccaaggaagacaaagggcaaatccagcggaaagggcaatgactcaacagagagagcggacgatggctcgagccaattaagagacaagcaaaacaataaggctggccccgccactacggagcctggcacatccaaccgagagcaatacaaagctcgaccaggtattgcatctgtgcagagggccactgaaagtgcagaaatgcccatgaagaataatgacgaagggacgccagataagaaaggaaatactaagggcgacctagttaatgagcatagtgaggctaaagacgaggcggatgaagcgacgaagaagcaggcaaaggatacagacaaaagtaaagcgcaagtcacatattcagacactggtatcaataatgctaatgaactgtcaagatctgggaatgtggataatgagggtggaagtaatcagaagccgatgtctaccagaatagctgaggcaacgtctgctatagtgtcgaaacatcctgcgcgtgttgggctgccacctaccgctagcagtggtcatgggtatcagtgccatgtctgttctgcagtcctgtttagtcctttagacctagatgcccacgtcgcctcacatggtttgcatggtaacatgacattaacatcgagtgatatccagcgacatataactgagttcatcagctcatggcaaaatcatcctattgttcaagtttcggctgatgtcgaaaataagaaaactgctcaattgcttcacgctgacactcctcgactcgtcacttgggatgctggtttgtgtacttcattcaaaatcgtcccgattgtgccagctcaggtgccgcaggatgtactggcctatacgtttttcacctcttcatacgctatccaatcaccgtttccagaggcggcagtgtctaggattgtggtgcatacgagatgggcatctaatgttgactttgaccgagactcgtctgtcatcatggcgccacctacagaaaacaatatccatttgtttaaacagttactaaatactgaaaccctgtctgtaaggggggctaatccgctaatgttcagggcgaatgtgttgcatatgttgctagagttcgtattagataacttgtatctgaacagacatacgggattctctcaagaccacacgccatttactgagggtgctaatttgcgttcacttcctggccccgatgctgagaaatggtactcgattatgtatccaacgcgcatgggaacgccgaatgtatccaaaatatgtaatttcgtcgcctcttgtgtgcgaaatcgggttggacggtttgatcgagcacagatgatgaacggagctatgtcagagtgggtggatgtcttcgagacttcagacgcgctaaccgtctccattcgaggtcgatggatggctagactagctcgcatgaacataaatccaacagagatcgaatgggcattgactgaatgtgcacaaggatatgtgactgtcacaagtccttacgctcctagcgtaaatagattgatgccctatcgtatctccaacgctgagcggcaaatatcacagataatcaggatcatgaacattggcaataacgcgacggtgatacaacctgttctgcaagatatttcggtactccttcaacgcatatcaccactccaaatagatccaactattatttccaacactatgtcaacagtctcggagtctactactcagaccctcagccccgcgtcctcaattttgggtaaactacgaccaagcaactcagatttttctagttttagagtcgcgttggctggatggctttataatggggttgtgacgacggtgattgatgatagttcatatccaaaagacggcggcagcgtgacctcacttgaaaatctgtgggatttcttcatccttgcgcttgctctaccactgacaactgacccctgtgcacctgtgaaagcattcatgaccctagccaacatgatggttggtttcgagacaatccctatggataatcagatctatactcaatcgagacgcgcgagtgctttctcaacgcctcacacgtggccacgatgctttatgaacatccagttaatttctccaatcgacgctcccatcttgcgacagtgggctgaaattattcatagatactggcctaacccttcacagattcgttatggtgcaccgaacgttttcggctcggcaaatttgttcactccacctgaggtgctgttattgccaatcgatcatcaaccagctaatgtaacaacgccaacgctggacttcaccaatgagttaactaattggcgcgctcgtgtctgtgagcttatgaagaatctcgttgataaccaaagatatcaacctggatggacacaaagtctagtctcgtcaatgcgcggaacgctagacaaattgaagttgattaaatcgatgacaccaatgtatctgcaacagctggctccggtagagttagcagtgatagctcccatgttgccttttccacctttccaggtgccatacgtccgtctcgatcgtgacagagttccaacaatggttggagtaacacgacattcacgagatactattactcagccggcgctatcgctgtcgacaaccaatactactgttggcgtgccactagctctagacgcgagggctatcaccgttgcgctgttgtcagggaaatatccgccggatttggtgacaaatgtatggtacgctgatgccatttacccaatgtatgcagacacggaggtgttctctaatcttcagagagacatgattacctgcgaggccgtgcagacattagtgactctggtggcgcaaatatcagagacccagtatcctgtagataggtatcttgattggatcccatcactgagagcatcggcggcgacggcggcgacatttgctgagtgggttaatacttcaatgaagacggcgtttgatttgtctgatatgctgttagagcctctcctaagcggtgatccgaggatgactcaactagcgattcagtatcagcagtacaatggcagaacgtttaatatcatacctgaaatgccaggttcagtaattgctgactgcgttcaattaacagcagaagtctttaatcacgaatataacctgtttgggattgcgcggggtgatatcatcattggccgtgttcagtcgacacatttgtggtcaccgctggctcctccacctgacctggtgtttgatcgtgatacccctggtgttcacatcttcggacgagattgccgtatatcgtttggaatgaatggcgccgcgccaatgattagagatgagactggactgatggtgccttttgaaggaaattggattttcccactggcgctttggcaaatgaatacacgatattttaatcaacagttcgacgcgtggattaagacaggagagttgcgaatccgcattgagatgggcgcgtatccatatatgttgcattactatgatccacgtcagtacgctaatgcatggaatttaacatccgcctggcttgaagaaattacgccgacgagcatcccatccgtgcctttcatggtgcccatttcaagtgatcatgacatttcctctgccccagctgtccaatatatcatttcaactgaatataatgatcggtctctgttctgcactaactcatcatctccccaaaccatcgctggaccagacaaacacattccagttgagagatataacattctgaccaaccccgacgctccacccacgcagatacaactgcctgaagtcgttgacttgtacaacgtcgtcacacgctatgcgtatgagactccgcctattaccgctgttgttatgggtgttccttgatcctcatcctcccaacaggtgctagagcattgcgctcaatgctagttgggccgattcatc

SEQ ID NO: 11 - protein, 455 aa, Reovirus
MDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 12 - protein, 418 aa, Reovirus
MARAAFLFKTVGFGGLQNVPINDELSSHLLRAGNSPWQLTQFLDWISLGRGLATSALVPTAGSRYYQMSCLLSGTLQIPFRPNHRWGDIRFLRLVWSAPTLDGLVVAPPQVLAQPALQAQADRVYDCDDYPFLARDPRFKHRVYQQLSAVTLLNLTGFGPISYVRVDEDMWSGDVNQLLMNYFGHTFAEIAYTLCQASANRPWEYDGTYARMTQIVLSLFWLSYVGVIHQQNTYRTFYFQCNRRGDAAEVWILSCSLNHSAQIRPGNRSLFVMPTSPDWNMDVNLILSSTLTGCLCSGSQLPLIDNNSVPAVSRNIHGWTGRAGNQLHGFQVRRMVTEFCDRLRRDGVMTQAQQNQVEALADQTQQFKRDKLETWAREDDQYNQAHPNSTMFRTKPFTNAQWGRGNTGATSAAIAALI

SEQ ID NO: 13 - protein, 254 aa, Reovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSNHQASTMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDMDVRWTRIAGRLVIRVWTLHAASTSCIARRQQKPSVCLRHALC

SEQ ID NO: 14 - protein, 366 aa, Reovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSNHQASTMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDISYGCEVDANSRPAGDQSMDSSRCINELYCEETAEAICVLKTCLVLNCMQFKLEMDDLAHNAAELDKIQMMIPFSERVFRMASSFATIDAQCFRFCVMMKDKNLKIDMRETTRLWTRSASDDSVATSSLSISLDRGRWVAADASDARLLVFPIRV

SEQ ID NO: 15 - protein, 365 aa, Reovirus
MEVCLPNGHQVVDLINNAFEGRVSIYSAQEGWDKTISAQPDMMVCGGAVVCMHCLGVVGSLQRKLKHLPHHRCNQQIRHQDYVDVQFADRVTAHWKRGMLSFVAQMHEMMNDVSPDDLDRVRTEGGSLVELNRLQVDPNSMFRSIHSSWTDPLQVVDDLDTKLDQYWTALNLMIDSSDLIPNFMMRDPSHAFNGVKLKGDARQTQFSRTFDSRSSLEWGVMVYDYSELDHDPSKGRAYRKELVTPARDFGHFGLSHYSRATTPILGKMPAVFSGMLTGNCKMYPFIKGTAKLKTVRKLVEAVNHAWGVEKIRYALGPGGMTGWYNRTMQQAPIVLTPAALTMFPDTIKFGDLNYPVMIGDPMILG

SEQ ID NO: 16 - protein, 736 aa, Reovirus
MAYIAVPAVVDSRSSEAIGLLESFGVDAGADANDVSYQDHDYVLDQLQYMLDGYEAGDVIDALVHKNWLHHSVYCLLPPKSQLLEYWKSNPSAIPDNVDRRLRKRLMLKKDLRKDDEYNQLARAFKISDVYAPLISSTTSPMTMIQNLNRGEIVYTTTDRVIGARILLYAPRKYYASTLSFTMTKCIIPFGKEVGRVPHSRFNVGTFPSIATPKCFVMSGVDIESIPNEFIKLFYQRVKSVHANILNDISPQIVSDMINRKRLRVHTPSDRRAAQLMHLPYHVKRGASHVDVYKVDVVDMLFEVVDVADGLRNVSRKLTMHTVPVCILEMLGIEIADYCIRQEDGMLTDWFLLLTMLSDGLTDRRTHCQYLINPSSVPPDVILNISITGFINRHTIDVMPDIYDFVKPIGAVLPKGSFKSTIMRVLDSISILGIQIMPRAHVVDSDEVGEQMEPTFEQAVMEIYKGIAGVDSLDDLIKWVLNSDLIPHDDRLGQLFQAFLPLAKDLLAPMARKFYDNSMSEGRLLTFSHADSELLNANYFGHLLRLKIPYITEVNLMIRKNREGGELFQLVLSYLYKMYATSAQPKWFGSLLRLLICPWLHMEKLIGEADPASTSAEIGWHIPREQLMQDGWCGCEDGFIPYVSIRAPRLVIEELMEKNWGQYHAQVIVTDQLVVGEPRRVSAKAVIKGNHLPVKLVSRFACFTLTAKYEMRLSCGHSTGRGAAYSARLAFRSDLA

SEQ ID NO: 17 - protein, 708 aa, Reovirus
MGNASSIVQTINVTGDGNVFKPSAETSSTAVPSLSLSPGMLNPGGVPWIAVGDETSVTSPGALRRMTSKDIPDTAIINTDNSSGAVPSESALVPYIDEPLVVVTEHAITNFTKAEMALEFNREFLDKMRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDSLIRRYPKEAAVALAKRNGGIQWMDVSEGTVMNEAVNAVAASALAPSASAPPLEEKSKLTEQAMDLVTAAEPEIIASLAPVPAPVFAIPPKPADYNVRTLRIDEATWLRMIPKSMNTPFQIQVTDNTGTNWHLNLRGGTRVVNLDQIAPMRFVLDLGGKSYKETSWDPNGKKVGFIVFQSKIPFELWTAASQIGQATVVNYVQLYAEDSSFTAQSIIATTSLAYNYEPEQLNKTDPEMNYYLLATFIDSAAITPTNMTQPDVWDALLTMSPLSAGEVTVKGAVVSEVVPADLIGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQNWTQGFLDKVSAHFPAPKPDCPTSGDSGESSNRRVKRDSYAGVVKRGYTR

SEQ ID NO: 18 - protein, 721 aa, Reovirus
MASFKGFSANTVPVSKAKRDISSLAATPGLRSQSFTPSVDMSQSREFLTKAIEQGSMSIPYQHVNVPKVDRKVVSLVVRPFSSGAFSISGVISPAHAYLLECLPQLEQAMAFVASPESFQASDVAKRFAIKPGMSLQDAITAFINFVSAMLKMTVTRQNFDVIVAEIERLASTSVSVRTEEAKVADEELMLFGLDHRGPQQLDVSDAKGIMKAADIQTTHDVHLAPGVGNIDPEIYNEGRFMFMQHKPLAADQSYFTLETADYFKIYPTYDEHDGRMADQKQSGLILCTKDEVLAEQTIFKLDAPDDKTVHLLDRDDDHVVARFTKVFIEDVAPGHHAAQRSGQRSVLDDLYANTQVISITSAALKWVVKHGVSDGIVNRKNVKVCVGFDPLYTLSTHNGVSLCALLMDEKLSVLNSACRMTLRSLMKTGRDVDAHRAFQRVLSQGYTSLMCYYHPSRKLAYGEVLFLERSNDVTDGIKLQLDASRQCHECPVLQQKVVELEKQIIMQKSIQSDPTPVALQPLLSQLRELSSEVTRLQMELSRAQSLNAQLEADVKSAQSCSLDMYLRHHTCINGHAKEDELLDAVRVAPDVRREIMEKRSEVRQGWCERISKEAAAKCQTVIDDLTLMNGKQAQEITELRDSAEKYEKQIAELVSTITQNQITYQQELQALVAKNVELDALNQRQAKSLRITPSLLSATPIDSVDDVADLIDFSVPTDEL

SEQ ID NO: 19 - protein, 1267 aa, Reovirus
MSSMILTQFGPFIESISGITDQSNDVFEDAAKAFSMFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVAEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYAWFILKTKSIFPQNTLHSMYESLEGGYCPNLEWLEPRSDYKFMYMGVMPLSAKYARSAPSNDKKARELGEKYGLSSVVGELRKRTKTYVKHDFASVRYIRDAMACTSGIFLVRTPTETVLQEYTQSPEIKVPIPQKDWTGPIGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSTTSGSAVIEKVIPLGVYASSPPNQSINIDISACDASITWDFFLSVIMAAIHEGVASSSIGKPFMGVPASIVNDESVVGVRAARPISGMQNMIQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGTTAGKVNSETIQKMLELISKYGEEFGWKYDIAYDGTAEYLKLYFIFGCRIPNLSRHPIVGKERANSSAEEPWPAILDQIMGVFFNGVHDGLQWQRWIRYSWALCCAFSRQRTMIGESVGYLQYPMWSFVYWGLPLVKAFGSDPWIFSWYMPTGDLGMYSWISLIRPLMTRWMVANGYVTDRCSPVFGNADYRRCFNELKLYQGYYMAQLPRNPKKSGRAAPREVREQFTQALSDYLLQNPELKSRVLRGRSEWEKYGAGIIHNPPSLFDVPHKWYQGAQEAAIATREELAEMDETLMRARRHRYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVNTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 20 - protein, 1289 aa, Reovirus
MANVWGVRLADSLSSPTIETRTRQYTLHDLCSDLDANPGREPWKPLRNQRTNNIVAVQLFRPLQGLVLDTQLYGFPGAFDDWERFMREKLRVLKYEVLRIYPISNYSNEHVNVFVANALVGAFLSNQAFYDLLPLLIINDTMIGDLLGTGASLSQFFQSHGDVLEVAAGRKYLQMENYSNDDDDPPLFAKDLSDYAKAFYSDTYEVLDRFFWTHDSSAGVLVHYDKPTNGHHYLLGTLTQMVSAPPYIINATDAMLLESCLEQFSANVRARPAQPVTRLDQCYHLRWGAQYVGEDSLTYRLGVLSLLATNGYQLARPIPRQLTNRWLSSFVSQIMSDGVNETPLWPQERYVQIAYDSPSVVDGATQYGYVRKNQLRLGMRISALQSLSDTPSPVQWLPQYTIDQAAMDEGDLMVSRLTQLPLRPDYGNIWVGDALSYYVDYNRSHRVVLSSELPQLPDTYFDGDEQYGRSLFSLARKIGDRSLVKDTAVLKHAYQAIDPNTGKEYLRSRQSVAYFGASAGHSGADQPLVIEPWIQGKISGVPPPSSVRQFGYDVARGAIVDLARPFPSGDYQFVYSDVDQVVDGHDDLSISSGLVESLLSSCMHATAPGGSFVVKINFPTRPVWHYIEQKILPNITSYMLIKPFVTNNVELFFVAFGVHQHSSLTWTSGVYFFLVDHFYRYETLSTISRQLPSFGYVDDGSSVTGIETISIENPGFSNMTQAARIGISGLCANVGNARKSIAIYESHGARVLTITSRRSPASARRKSRLRYLPLIDPRSLEVQARTILPADPVLFENVSGASPHVCLTMMYNFEVSSAVYDGDVVLDLGTGPEAKILELIPATSPVTCVDIRPTAQPSGCWNVRTTFLELDYLSDGWITGVRGDIVTCMLSLGAAAAGKSMTFDAAFQQLIKVLSKSTANVVLVQVNCPTDVVRSIKGYLEIDSTNKRYRFPKFGRDEPYSDMDALEKICRTAWPNCSITWVPLSYDLRWTRLALLESTTLSSASIRIAELMYKYMPIMRIDIHGLPMEKRGNFIVGQNCSLVIPGFNAQDVFNCYFNSALAFSTEDVNAAMIPQVSAQFDATKGEWTLDMVFSDAGIYTMQALVGSNANPVSLGSFVVDSPDVDITDAWPAQLDFTIAGTDVDITVNPYYRLMTFVRIDGQWQIANPDKFQFFSSASGTLVMNVKLDIADKYLLYYIRDVQSRDVGFYIQHPLQLLNTITLPTNEDLFLSAPDMREWAVKESGNTICILNSQGFVLPQDWDVLTDTISWSPSIPTYIVPPGDYTLTPL

SEQ ID NO: 21 - protein, 1275 aa, Reovirus
MKRIPRKTKGKSSGKGNDSTERADDGSSQLRDKQNNKAGPATTEPGTSNREQYKARPGIASVQRATESAEMPMKNNDEGTPDKKGNTKGDLVNEHSEAKDEADEATKKQAKDTDKSKAQVTYSDTGINNANELSRSGNVDNEGGSNQKPMSTRIAEATSAIVSKHPARVGLPPTASSGHGYQCHVCSAVLFSPLDLDAHVASHGLHGNMTLTSSDIQRHITEFISSWQNHPIVQVSADVENKKTAQLLHADTPRLVTWDAGLCTSFKIVPIVPAQVPQDVLAYTFFTSSYAIQSPFPEAAVSRIVVHTRWASNVDFDRDSSVIMAPPTENNIHLFKQLLNTETLSVRGANPLMFRANVLHMLLEFVLDNLYLNRHTGFSQDHTPFTEGANLRSLPGPDAEKWYSIMYPTRMGTPNVSKICNFVASCVRNRVGRFDRAQMMNGAMSEWVDVFETSDALTVSIRGRWMARLARMNINPTEIEWALTECAQGYVTVTSPYAPSVNRLMPYRISNAERQISQIIRIMNIGNNATVIQPVLQDISVLLQRISPLQIDPTIISNTMSTVSESTTQTLSPASSILGKLRPSNSDFSSFRVALAGWLYNGVVTTVIDDSSYPKDGGSVTSLENLWDFFILALALPLTTDPCAPVKAFMTLANMMVGFETIPMDNQIYTQSRRASAFSTPHTWPRCFMNIQLISPIDAPILRQWAEIIHRYWPNPSQIRYGAPNVFGSANLFTPPEVLLLPIDHQPANVTTPTLDFTNELTNWRARVCELMKNLVDNQRYQPGWTQSLVSSMRGTLDKLKLIKSMTPMYLQQLAPVELAVIAPMLPFPPFQVPYVRLDRDRVPTMVGVTRHSRDTITQPALSLSTTNTTVGVPLALDARAITVALLSGKYPPDLVTNVWYADAIYPMYADTEVFSNLQRDMITCEAVQTLVTLVAQISETQYPVDRYLDWIPSLRASAATAATFAEWVNTSMKTAFDLSDMLLEPLLSGDPRMTQLAIQYQQYNGRTFNIIPEMPGSVIADCVQLTAEVFNHEYNLFGIARGDIIIGRVQSTHLWSPLAPPPDLVFDRDTPGVHIFGRDCRISFGMNGAAPMIRDETGLMVPFEGNWIFPLALWQMNTRYFNQQFDAWIKTGELRIRIEMGAYPYMLHYYDPRQYANAWNLTSAWLEEITPTSIPSVPFMVPISSDHDISSAPAVQYIISTEYNDRSLFCTNSSSPQTIAGPDKHIPVERYNILTNPDAPPTQIQLPEVVDLYNVVTRYAYETPPITAVVMGVP

(19) United States
(12) Patent Application Publication
(10) Pub. No.: US 2011/0086005 A1
(43) Pub. Date: Apr. 14, 2011

(54) MODULATING INTERSTITIAL PRESSURE AND ONCOLYTIC VIRAL DELIVERY AND DISTRIBUTION

(75) Inventors: Matthew C. Coffey, Calgary (CA); Bradley G. Thompson, Calgary (CA); Hardev Pandha, Surrey (GB)

(73) Assignee: ONCOLYTICS BIOTECH INC., Calgary, AB (CA)

(21) Appl. No.: 12/994,119

(22) PCT Filed: May 27, 2009
(86) PCT No.: PCT/CA2009/000720

§ 371 Date: Nov. 22, 2010

Related U.S. Application Data
(60) Provisional application No. 61/056,276, filed on May 27, 2008.
(60) Provisional application No. 61/113,845, filed on Nov. 12, 2008.

(51) Int. Cl. A61K 38/20; A61K 35/76; A61K 38/21; A61P 43/00

(52) U.S. Cl. 424/85.2; 424/93.6; 424/85.4

(57) ABSTRACT

Provided herein are methods of treating a proliferative disorder in a subject comprising decreasing interstitial pressure and/or increasing vascular permeability in the subject and administering to the subject an oncolytic virus. Such methods improve oncolytic viral delivery and distribution.

### BACKGROUND

Oncolytic virus therapy is unique in the sense that, although it is a large molecule and is dependent upon solvent drag to assist effective delivery, these agents are able to replicate themselves and propagate in tumor targets, lyse target cells, release progeny and retarget adjacent cells. Thus, oncolytic viruses mitigate the total dependency on convection for delivery throughout the tumor mass.

### SUMMARY

Provided herein are methods of treating a proliferative disorder in a subject comprising decreasing interstitial pressure and/or increasing vascular permeability in the subject and administering to the subject an oncolytic virus. Such methods improve oncolytic viral delivery and distribution.

The details of one or more aspects are set forth in the accompanying drawings and the description below. Other features, objects, and advantages will be apparent from the description and drawings, and from the claims.

### BRIEF DESCRIPTION OF THE DRAWINGS

FIGS. 1A, 1B, and 1C are graphs showing the effect of reovirus and rapamycin on B16.F10 cells in vitro. Cells (5×10³ per well) were seeded in 96 well plates and allowed to adhere overnight. Culture medium was replaced with doubling dilutions of rapamycin and/or reovirus, corresponding to 2, 1, 0.5 and 0.25 times the previously determined ED50, diluted in fresh culture medium and incubation continued for 48 h. Medium was then removed and percentage cell survival compared to untreated cells was determined using the MTS assay.

FIGS. 2A and 2B are graphs showing reovirus and rapamycin are synergistic in vivo. B16.F10 tumors were seeded subcutaneously in C57B1/6 mice and treated with intratumoral reovirus T3D 5×10⁸ TCID50 on day 1 and 4, and intraperitoneal rapamycin 5 mg/kg on day 1, 4, 8 and 12 either alone or in combination, or with control treatment (intratumoral PBS, intraperitoneal PBS). FIG. 2A is a graph showing the average tumor diameter of B16.F10 tumors in C57B1/g mice treated with reovirus and rapamycin.

FIG. 2B is a graph showing the survival data for C57B1/g mice with B16.F10 tumors treated with reovirus and rapamycin.

FIG. 3 is a graph showing Treg depletion+IL-2 enhances systemic delivery of reovirus to subcutaneous tumors. C57B1/6 mice were seeded with subcutaneous B16 tumors. Nine days later, mice received an intraperitoneal injection of anti-CD25 antibody PC-61 or a control IgG. Twenty-four hours later, mice were injected intraperitoneally with PBS or with recombinant human IL-2 at a dose of 75,000 units/injection three times a day for 3 d. On the fourth day, a single further injection of IL-2 was given. Two hours after this last injection of IL-2/PBS, mice received an intravenous injection of reovirus (3.75×10⁹ TCID50) followed 24 h later by a second similar injection of virus. 72 h later, tumors were explanted and dissociated and viral titers recovered from freeze/thaw lysates of tumors from mice treated as shown were determined (3 mice per group).

FIGS. 4A and 4B are graphs showing CPA-mediated Treg modification, with IL-2 and lower-dose reovirus, is therapeutic against established tumors. For FIG. 4A, C57B1/6 mice were seeded with subcutaneous B16 tumors. Nine days later, mice received an intraperitoneal injection of either CPA (100 mg/kg) or anti-CD25 antibody PC-61or PBS. Twenty-four hours later, mice were injected intraperitoneally with PBS or with recombinant human IL-2 at a dose of 75,000 units/injection three times a day for 3 d. On the fourth day, a single further injection of IL-2 was given. Two hours after this last injection of IL-2/PBS, mice received an intravenous injection of reovirus at a lower than maximal achievable dose of 1×10⁸ TCID50 followed 24 h later by a second similar injection of virus. Survival of mice (tumor <1.0 cm in any diameter) with time after tumor seeding is shown (n=7 per group). The median survival times of groups treated with reovirus alone (median survival, 21d), CPA/IL-2 (23 d), PC-61/reovirus (22 d), or CPA/reovirus (21 d) were not significantly different from each other and none of these treatments generated any long-term survivors. Median survival times of groups treated with IL-2/reovirus (25 d), PC-61/IL-2/reovirus (24d), or CPA/IL-2/reovirus (25 d) were significantly longer (P=0.04) than these other groups. Treatment with PC-61/IL-2/reovirus or CPA/IL-2/reovirus led to long-term survivors and both of these were significantly more therapeutic. **, P<0.01. FIG. 4B is a graph showing neutralizing antibodies against reovirus in serum recovered from mice 7 to 10 days after the final viral injection of the mice as described in FIG. 4A.

### DETAILED DESCRIPTION

Large, biological agents for the treatment of neoplasia may be limited by intratumoral interstitial pressure and/or reduced vascular permeability. Further, diffusion seems to be the most important mode of passive transport of small molecules (i.e., MW 4000 Da) in tissues, whereas convection or solvent drag typically is the major mechanism of movement of large proteins (MW >40,000 Da).

Interstitial pressure within a tumor mass may be the result of increased microvascular pressure (MVP), which is dependent upon the arteriovenous pressure difference and geometric and viscous resistance to blood flow (i.e., the result of the decrease in vessel diameter which is a function of the physical stress induced on the vessel by the growth of solid tumors decreasing vessel diameter). As such, the intratumoral environment is one which results in increased interstitial pressure and/or decreased vascular permeability and may inhibit delivery of large molecules. Agents that decrease hydrostatic pressure in a tumor create a situation where the hydrostatic pressure outside of the tumor mass would be greater than that of the tumor itself. This situation aids in the delivery of large molecules, such as oncolytic viruses. Thus, provided herein are methods of treating a proliferative disorder in a subject comprising decreasing interstitial pressure and/or increasing vascular permeability in a subject in need of treatment and administering to the subject in need of treatment an oncolytic virus. Optionally, the oncolytic virus is administered at the same time, before or after decreasing interstitial pressure and/or increasing vascular permeability in the subject.

Optionally, the interstitial pressure in the subject is decreased by an agent that decreases interstitial pressure and/or increases vascular permeability. Thus, agents that decrease interstitial pressure, optionally, increase vascular permeability, as well. Alternatively, an agent that decreases interstitial pressure can be used in combination with an agent that increases vascular permeability.

Agents suitable for use in the provided methods include a taxane. Suitable taxanes for use in the provided methods include, but are not limited to, taxol (paclitaxel), larotaxel, and taxotere (docetaxel). Other agents include, but are not limited to, vasopressin; TNF; interleukin-1 (IL-1); interferon-K (IFN-K); substance P; proteinase inhibitors such as N-alpha-tosyl-L-lysyl-chloromethyl-ketone (TLCK), tosyl phenylalanyl chloromethyl ketone (TPCK) and leupeptin; vascular endothelial growth factor (VEGF); nitroglycerine; serotonin; plasma kinins such as bradykinin; platelet-activating factor (PAF); prostaglandin E₁ (PGE₁); histamine; imatinib; zona occludens toxin (ZOT); interleukin-2; nitric oxide inhibitors such as L-N-monomethyl arginine (L-NMMA) and L-N-nitro-arginine methyl ester (L-NAME); and human growth factor receptor tyrosine kinase inhibitors such as gefitinib. See Martin et al., Immunology 64(2):301-5 (1988); Zhou et al., Radiat. Res. 168(3):299-307 (2007); Watanabe et al., Inflammation Research 17(5-6):472-7 9 (1986); U.S. Publication No. 2005/0101559; Moasser et al., J. Magn. Reson. Imaging 26(6):1618-25 (2007); and Vlahovic et al., Br. J Cancer 97(6):735-40 (2007), which are incorporated herein by reference in their entireties at least for the agents described therein and methods of making and using the agents.

Optionally, the interstitial pressure in the subject is decreased by lowering extracellular calcium ion concentrations. Low extracellular calcium ion concentration conditions also can be used to enhance vascular permeability. For example, a low calcium ion concentration fluid can be perfused through the vasculature of the tissue to which the oncolytic virus is administered. Suitable perfusate calcium ion concentrations may range from about 40 or 50 Tmol/L to about 500 Tmol/L, more preferably from about 50 Tmol/L to about 200 Tmol/L. A perfusate calcium concentration of about 50 Tmol/L is provided. Calcium ion (e.g., Ca²⁺) concentration can also be lowered, for example, through use of a suitable buffer such as a chelating agent, for example, ethylenebis(oxyethylenenitrilo)tet-racetic acid (EGTA), ethylenediaminetetracetic acid (EDTA), or 1,2-bis-(2-aminophenoxy)ethane-N,N,N′,N′-tetraacetic acid (BAPTA). See U.S. Publication No. 2005/0101559, which is incorporated by reference herein in its entirety. Thus, provided herein are methods of treating a proliferative disorder in a subject comprising administering to the subject a low calcium ion concentration fluid that decreases interstitial pressure and an oncolytic virus. Optionally, the method further comprises administering an agent that increases vascular permeability.

Optionally, the interstitial pressure of a tumor can be reduced by removal of excess interstitial fluid. Removal of excess interstitial fluid is accomplished by any known method, including, for example, by an artificial lymphatic system (ALS). Such methods are described in, for example, U.S. Publication No. 2001/0047152; U.S. Pat. No. 5,484,399; U.S. Publication No. 2005/0165342; and U.S. Publication No. 2003/0149407, which are incorporated by reference herein, in their entireties. Thus, provided herein are methods of treating a tumor in a subject comprising reducing in the subject the excess interstitial fluid of a tumor and administering to the subject an oncolytic virus. Optionally, the excess interstitial fluid is removed prior to administration of the oncolytic virus. Optionally, the method further comprises administering an agent that increases vascular permeability.

If the oncolytic virus is administered systemically, permeabilizing photodynamic therapy (P-PDT) can be used to enhance delivery of the oncolytic virus by enhancing vascular permeability. P-PDT induced vascular leakiness allows the therapeutic agents to leave the vasculature and distribute into hyperproliferative tissue (e.g. the tumor bed) in higher concentrations than achievable without prior permeabilizing PDT. See U.S. Publication No. 2004/0010218, which is incorporated by reference herein in its entirety. Thus, provided herein are methods of treating a proliferative disorder in a subject comprising administering to the subject a permeabilizing photodynamic therapeutic agent and an oncolytic virus. Optionally, the permeabilizing photodynamic therapeutic agent is administered prior to administration of the oncolytic virus. Optionally, the method further comprises administering an agent that decreases interstitial pressure.

Optionally, the provided methods further comprise administering to the subject an immunosuppressive agent. Optionally, the immunosuppressive agent is an agent that inhibits a pro-inflammatory cytokine. As used herein, a pro-inflammatory cytokine refers to a cytokine that directly or indirectly stimulates the immune system. Pro-inflammatory cytokines include, but are not limited to, IL-1I, IL-3, IL-6, IL-12 p70, IL-17, MIP-1I, and RANTES. Thus, provided herein are methods of treating a proliferative disorder in a subject comprising administering to the subject in need of treatment, an agent that decreases interstitial pressure, an agent that inhibits a pro-inflammatory cytokine and an oncolytic virus. Optionally, the agent that decreases interstitial pressure is administered to the subject first, followed by administration of the agent that inhibits a pro-inflammatory cytokine and the oncolytic virus. Optionally, the oncolytic virus is then administered after the agent that inhibits the pro-inflammatory cytokine. The agent that inhibits the pro-inflammatory cytokine, optionally, inhibits the expression or activity of the pro-inflammatory cytokine. Optionally, the agent blocks T-cell responses while having little to no effect on B-cell activity. Thus, the agent inhibits pro-inflammatory cytokines but does not inhibit or minimally inhibits production of NARA. Optionally, the agent is a platinum compound. Suitable platinum compounds also include, but are not limited to, cisplatin, carboplatin, metaplatin, and oxaliplatin. Optionally, the agent that decreases interstitial pressure is paclitaxel, the agent that inhibits a pro-inflammatory cytokine is carboplatin and the oncolytic virus is a reovirus.

Other agents that inhibit pro-inflammatory cytokines include, but are not limited to, TNF-I antibodies such as infliximab, CDP571, CDP870, and adalimumab; recombinant, human soluble p55 TNF receptors such as onercept; soluble TNF receptor and Fc fragment fusion proteins such as etanercept; pegylated Fab fragments of humanized antibody to TNF such as certolizumab pegol; chimeric antibodies to anti-I chain of IL-2 receptor such as basiliximab or daclizumab; IL-12p40 antibodies such as ABT-874; IL-6 receptor antibodies such as MRA or tocilizumab; IFN-K antibodies such as fontolizumab; antibodies that inhibit IL-1 binding to the IL-1 receptor such as AMG108; caspase-1 inhibitors that inhibit cytokine-release such as diarylsulphonylurene; IL-15 antibodies such as mepolizumab; IL-8 antibodies such as ABX-IL-8; IL-9 antibodies including IL-9 monoclonal antibodies; recombinant human IL-21 also referred to as 494C 10; inhibitors of TNF-I, IL-18, IL-6 and granulocyte monocyte-colony stimulating factor expression such as biophylum sensitivum; NF-PB signaling blockers that inhibit pro-inflammatory cytokine expression such as simvastatin; and inhibitors of IL-6 expression and NF-PB activation such as (−)-epigallocatechin-3-gallate (EGCG).

Other agents that inhibit pro-inflammatory cytokines include human recombinant lactoferrin, which inhibits cellular release of proinflammatory cytokines and prometastatic cytokines (including IL-6, IL-8, granulocyte macrophage colony-stimulating factor and TNF-a). Inhibitors of dendritic cell derived IL-12 and IL-18, such as rapamycin and sanglifehrin, are also suitable for use in the provided methods. Rapamycin is an immunosuppressant that inhibits T cell mTOR kinase activation, and Sanglifehrin A is a cyclophilin-binding immunosuppressant that also inhibits IL-2 dependent T cell proliferation. Also suitable for use in the provided methods is dietary rutin, which suppresses the induction of pro-inflammatory cytokines such as IL-1β, IL-6, and GM-CS.

Optionally, the provided methods further include the step of selecting a subject with a proliferative disorder. Thus, provided is a method of treating a proliferative disorder in a subject comprising selecting a subject with a proliferative disorder, administering to the subject in need of treatment an agent that decreases interstitial pressure and an oncolytic virus. Optionally, the proliferative disorder is a ras-mediated proliferative disorder. Thus, the provided methods, optionally, further comprise the step of selecting a subject with a ras-mediated proliferative disorder. Optionally, the proliferative disorder is a proliferative disorder characterized by interferon-resistance, p53-deficiency or Rb-deficiency.

Optionally, the subject is in need of enhanced delivery of an oncolytic virus. Thus, provided herein are methods of enhancing delivery of an oncolytic virus to a subject with a proliferative disorder comprising administering to the subject an agent that decreases interstitial pressure and administering to the subject the oncolytic virus. Such methods can also comprise the step of selecting a subject with a proliferative disorder.

Optionally, the provided methods comprise the step of diagnosing the phenotype of the proliferative disorder, for example, by determining whether the proliferative disorder is a ras-mediated proliferative disorder. By way of another example, the provided methods comprise the step of determining whether the proliferative disorder is an interferon-resistant tumor, p53 deficient tumor or an Rb-deficient tumor. Such methods for determining whether a proliferative disorder has a certain phenotype are known. See, for example, U.S. Pat. No. 7,306,902, which is incorporated herein by reference in its entirety.

Oncolytic viruses that are used in the provided methods include, but are not limited to, oncolytic viruses that are members in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hepadnaviridae, retroviridae, cyctoviridae, reoviridae, birnaviridae, paramyxoviridae, rhabdoviridae, filoviridae, orthomyxoviridae, bunyaviridae, arenaviridae, leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. Immunoprotected viruses and reassortant or recombinant viruses of these and other oncolytic viruses are also encompassed by the provided methods. Furthermore, a combination of at least two oncolytic viruses can also be employed to practice the provided methods. A few oncolytic viruses are discussed below, and a person of ordinary skill in the art can practice the present methods using additional oncolytic viruses as well according to the disclosure herein and knowledge available in the art.

Normally, when a virus enters a cell, double-stranded RNA Kinase (PKR) is activated, blocking protein synthesis, and the virus cannot replicate in this cell. Some viruses have developed a system to inhibit PKR and facilitate viral protein synthesis as well as viral replication. For example, adenovirus makes a large amount of a small RNA, VA1 RNA. VA1 RNA has extensive secondary structures and binds to PKR in competition with the double-stranded RNA (dsRNA) which normally activates PKR. Since it requires a minimum length of dsRNA to activate PKR, VA1 RNA does not activate PKR. Instead, it sequesters PKR by virtue of its large amount. Consequently, protein synthesis is not blocked, and adenovirus can replicate in the cell.

Ras-activated neoplastic cells are not subject to protein synthesis inhibition by PKR because ras inactivates PKR. These cells are therefore susceptible to viral infection even if the virus does not have a PKR-inhibitory system. Accordingly, if the PKR inhibitors in adenovirus, vaccinia virus, herpes simplex virus, or parapoxvirus orf virus are mutated so as not to block PKR function anymore, the resulting viruses do not infect normal cells due to protein synthesis inhibition by PKR, but they replicate in ras-activated neoplastic cells which lack PKR activities. By way of example, reoviruses selectively replicate and lyse ras-activated neoplastic cells.

Accordingly, a virus, modified or mutated such that it does not inhibit PKR function, selectively replicates in ras-activated neoplastic cells while normal cells are resistant. Optionally, the oncolytic virus is an adenovirus mutated in the VA1 region, a vaccinia virus mutated in the K3L and/or E3L region, a vaccinia virus mutated in the thymidine kinase (TK) gene, a vaccinia virus mutated in the vaccinia growth factor (VGF) gene, a herpes virus mutated in the y134.5 gene, a parapoxvirus orf virus mutated in the OV20.0L gene, or an influenza virus mutated in the NS-1 gene.

Vaccinia viruses mutated in the viral thymidine kinase (TK) gene are unable to make nucleotides needed for DNA replication. In normal cells, the cellular TK levels are usually very low and the virus is unable to replicate. In tumors, loss of the tumor suppressor Rb or an increase in cyclin activity, leads to E2F pathway activation and high levels of TK expression. Thus, cancer cells have high TK levels and the mutated vaccinia virus can replicate and spread.

The vaccinia growth factor (VGF) gene is a homolog of mammalian epidermal growth factor (EGF) and can bind and activate the EGF Receptor (EGFR). Vaccinia viruses mutated in the VGF gene are growth restricted to cells with activated EGF pathways, which is commonly mutated in cancers.

The viruses can be modified or mutated according to the known structure-function relationship of the viral PKR inhibitors. For example, since the amino terminal region of E3 protein interacts with the carboxy-terminal region domain of PKR, deletion or point mutation of this domain prevents anti-PKR function (Chang et al., PNAS 89:4825-4829 (1992); Chang, H. W. et al., Virology 194:537-547 (1993); Chang et al., J. Virol. 69:6605-6608 (1995); Sharp et al., Virol. 250:301-315 (1998); and Romano et al., Mol. and Cell. Bio. 18:7304-7316 (1998)). The K3L gene of vaccinia virus encodes pK3, a pseudosubstrate of PKR. Truncations or point mutations within the C-terminal portion of K3L protein that is homologous to residues 79 to 83 in eIF-2 abolish PKR inhibitory activity (Kawagishi-Kobayashi, M., et al., Mol. Cell. Biology 17:4146-4158 (1997)).

Another example is the Delta24 virus, which is a mutant adenovirus carrying a 24 base pair deletion in the ElA region (Fueyo, J., et al., Oncogene 19(1):2-12 (2000)). This region is responsible for binding to the cellular tumor suppressor Rb and inhibiting Rb function, thereby allowing the cellular proliferative machinery, and hence virus replication, to proceed in an uncontrolled fashion. Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell.

In addition, vesicular stomatitis virus (VSV) selectively kills neoplastic cells (and interferon can be added). A herpes simplex virus 1 (HSV-1) mutant defective in ribonucleotide reductase expression, hrR3, replicates in colon carcinoma cells but not normal liver cells (Yoon, S. S., et al., FASEB J. 14:301-311(2000)). Newcastle disease virus (NDV) replicates preferentially in malignant cells, and the most commonly used strain is 73-T (Reichard, K. W., et al., J. of Surgical Research 52:448-453 (1992); Zorn, U. et al., Cancer Biotherapy 9(3):22-235 (1994); Bar-Eli, N., et al., J. Cancer Res. Clin. Oncol. 122: 409-415 (1996)). Vaccinia virus propagates in several malignant tumor cell lines. Encephalitis virus has an oncolytic effect in a mouse sarcoma tumor, but attenuation may be required to reduce its infectivity in normal cells. Tumor regression has been described in tumor patients infected with herpes zoster, hepatitis virus, influenza, varicella, and measles virus (for a review, see Nemunaitis, J., Invest. New

Drugs 17:375-386 (1999)).

Optionally, the oncolytic virus is a reovirus. Reovirus refers to any virus classified in the reovirus genus, whether naturally occurring, modified, or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reoviruses have been recovered from many species. All three types share a common complement-fixing antigen.

The human reovirus includes three serotypes: type 1 (strain Lang or T1L), type 2 (strain Jones, T2J), and type 3 (strain Dearing or strain Abney, T3D). The three serotypes are easily identifiable on the basis of neutralization and hemagglutinin-inhibition assays. A reovirus according to this disclosure can be a type 3 mammalian orthoreovirus. Type 3 mammalian orthoreoviruses include, without limitation, Dearing and Abney strains (T3D or T3A, respectively). See, for example, ATCC Accession Nos. VR-232 and VR-824. As described previously, reoviruses use a host cell's ras pathway machinery to downregulate double-stranded RNA-activated protein kinase (PKR) and thus replication in the cell. See, for example, U.S. Pat. Nos. 6,110,461; 6,136,307; 6,261,555; 6,344,195; 6,576,234; and 6,811,775, which are incorporated by reference herein in their entireties.

The reovirus may be naturally occurring or modified. The reovirus is naturally-occurring when it can be isolated from a source in nature and has not been intentionally modified by humans in the laboratory. For example, the reovirus can be from a field source, that is, from a human who has been infected with the reovirus. The reovirus may also be selected or mutagenized for enhanced oncolytic activity.

The reovirus may be modified but still capable of lytically infecting a mammalian cell having an active ras pathway. The reovirus may be chemically or biochemically pretreated (e.g., by treatment with a protease, such as chymotrypsin or trypsin) prior to administration to the proliferating cells. Pretreatment with a protease removes the outer coat or capsid of the virus and may increase the infectivity of the virus. The reovirus may be coated in a liposome or micelle (Chandran and Nibert, J. of Virology 72(1):467-75 1998). For example, the virion may be treated with chymotrypsin in the presence of micelle-forming concentrations of alkyl sulfate detergents to generate a new infectious subviral particle (ISVP).

The reovirus may be a recombinant reovirus. For example, the recombinant reovirus can be a reassortant reovirus, which includes genomic segments from two or more genetically distinct reoviruses. Recombination/reassortment of reovirus genomic segments may occur following infection of a host organism with at least two genetically distinct reoviruses. Recombinant/reassortant viruses can also be generated in cell culture, for example, by co-infection of permissive host cells with genetically distinct reoviruses.

Accordingly, the provided methods include the use of a recombinant reovirus resulting from reassortment of genome segments from two or more genetically distinct reoviruses, including but not limited to, human reovirus, such as type 1 (e.g., strain Lang), type 2 (e.g., strain Jones), and type 3 (e.g., strain Dearing or strain Abney); non-human mammalian reoviruses; or avian reovirus. Optionally, the provided methods include the use of recombinant reoviruses resulting from reassortment of genome segments from two or more genetically distinct reoviruses wherein at least one parental virus is genetically engineered, comprises one or more chemically synthesized genomic segment, has been treated with chemical or physical mutagens, or is itself the result of a recombination event. Optionally, the provided methods include the use of the recombinant reovirus that has undergone recombination in the presence of chemical mutagens, including but not limited to, dimethyl sulfate and ethidium bromide, or physical mutagens, including but not limited to, ultraviolet light and other forms of radiation.

Optionally, the provided methods include the use of reoviruses with mutations (including insertions, substitutions, deletions or duplications) in one or more genome segments. Such mutations can comprise additional genetic information as a result of recombination with a host cell genome or can comprise synthetic genes. For example, mutant reoviruses as described herein can contain a mutation that reduces or essentially eliminates expression of a sigma3 polypeptide or that results in the absence of a functional sigma3 polypeptide as described in U.S. Ser. No. 12/124,522, which is incorporated by reference herein in its entirety. A mutation that eliminates expression of a sigma3 polypeptide or that results in the absence of a functional sigma3 polypeptide can be in the nucleic acid encoding the sigma3 polypeptide (i.e., the S4 gene) or in a nucleic acid that encodes a polypeptide that regulates the expression or function of the sigma3 polypeptide.

As used herein, a mutation that reduces the expression of a sigma3 polypeptide refers to a mutation that results in a decrease in the amount of sigma3 polypeptides, compared to a reovirus expressing wild type levels of sigma3 polypeptide, of at least 30% (e.g., at least 40%, 50%, 60%, 70%, 80%, 90%, or 95%). As used herein, a mutation that essentially eliminates expression of a sigma3 polypeptide refers to a mutation that results in a decrease in the amount of sigma3 polypeptides, relative to the amount of sigma3 polypeptides produced by a wild type reovirus, of at least 95% (e.g., 96%, 97%, 98%, 99%, or 100%). As used herein, a mutation that results in a decrease in or absence of a functional sigma3 polypeptide refers to a mutation that allows expression of the sigma3 polypeptide but that results in a sigma3 polypeptide that is not able to assemble or incorporate into the viral capsid. It would be understood that it may be desirable or necessary for sigma3 polypeptides to retain other functionalities (e.g., the ability to bind RNA) in order that the mutant reovirus retain the ability to propagate.

A mutation in a sigma3 polypeptide as described herein can result in a sigma3 polypeptide that is incorporated into the capsid at levels that are reduced relative to a sigma3 polypeptide that does not contain the mutation (e.g., a wild type sigma3 polypeptide). A mutation in a sigma3 polypeptide as described herein also can result in a sigma3 polypeptide that cannot be incorporated into a viral capsid. Without being bound by any particular mechanism, a sigma3 polypeptide may have reduced function or lack function due, for example, to an inability of the sigma3 polypeptide and the mul polypeptide to bind appropriately, or due to a conformational change that reduces or prohibits incorporation of the sigma3 polypeptide into the capsid.

In addition to a mutation that abolishes or reduces expression of the sigma3 polypeptide or that results in a non-functional or reduced-function sigma3 polypeptide, a mutant reovirus as described herein also can contain one or more further mutations (e.g., a second, third, or fourth mutation) in one of the other reovirus capsid polypeptides (e.g., mul, lambda2, and/or sigmal). Reoviruses containing a mutation affecting the sigma3 polypeptide and, optionally, a further mutation in any or all of the other outer capsid proteins can be screened for the ability of such mutant reoviruses to infect and cause lysis of cells. For example, neoplastic cells that are resistant to lysis by wild type reovirus can be used to screen for effective mutant reoviruses described herein.

For example, a further mutation can reduce or essentially eliminate expression of a mul polypeptide or result in the absence of a functional mul polypeptide. The mul polypeptide, which is encoded by the M2 gene, is likely involved in cell penetration and may play a role in transcriptase activation. Each virion contains about 600 copies of mul polypeptides, which are present in the form of 1:1 complexes with sigma3 polypeptides. The mul polypeptide is myristolated on its N-terminus, and then the myristolated N-terminal 42 residues are cleaved off, resulting in a C-terminal fragment (mulC). Additionally or alternatively, a further mutation can reduce or essentially eliminate expression of a lambda2 polypeptide or result in the absence of a functional lambda2 polypeptide, and/or a further mutation can reduce or essentially eliminate expression of a sigmal polypeptide or result in the absence of a functional sigmal polypeptide. The lambda2 polypeptide is encoded by the L2 gene, is involved in particle assembly, and exhibits guanylyltransferase and methyltransferase activity. The sigmal polypeptide is encoded by the Si gene, is involved in cell-attachment and serves as the viral hemagglutinin.

For example, the reovirus has a lambda-3 polypeptide having one or more amino acid modifications; a sigma-3 polypeptide having one or more amino acid modifications; a mu-1 polypeptide having one or more amino acid modifications; and/or a mu-2 polypeptide having one or more amino acid modifications, as described in U.S. Ser. No. 12/046,095, which is incorporated by reference herein in its entirety. By way of example, the one or more amino acid modifications in the lambda-3 polypeptide are a Val at residue 214, an Ala at residue 267, a Thr at residue 557, a Lys at residue 755, a Met at residue 756, a Pro at residue 926, a Pro at residue 963, a Leu at residue 979, an Arg at residue 1045, a Val at residue 1071, or any combination thereof, numbered relative to GenBank Accession No. M24734.1. It is noted that, when the amino acid sequence is a Val at residue 214 or a Val at residue 1071, the amino acid sequence further includes at least one additional change in the amino acid sequence. Optionally, the lambda-3 polypeptide includes the sequence shown in SEQ ID NO:18. Further by way of example, the one or more amino acid modifications in the sigma-3 polypeptide are a Leu at residue 14, a Lys at residue 198, or any combination thereof, numbered relative to GenBank Accession No. K02739. It is noted that, when the amino acid sequence is a Leu at residue 14, the amino acid sequence further includes at least one additional change in the amino acid sequence. Optionally, the sigma-3 polypeptide includes the sequence shown in SEQ ID NO:14. Further by way of example, the one or more amino acid modifications in the mu-1 polypeptide is an Asp at residue 73 numbered relative to GenBank Accession No. M20161.1. Optionally, the mu-1 polypeptide includes the sequence shown in. SEQ ID NO:16. Also by way of example, the amino acid modification mu-2 polypeptide is a Ser at residue 528 numbered relative to GenBank Accession No. AF461684.1. Optionally, the mu-1 polypeptide includes the sequence shown in SEQ ID NO:15. A reovirus as described herein having one or more modifications can further include a reovirus sigma-2 polypeptide. Such a sigma-2 polypeptide has a Cys at one or more of position 70, 127, 195, 241, 255, 294, 296, or 340, numbered relative to GenBank Accession No. NP_{—}694684.1. Optionally, the sigma-2 polypeptide includes the sequence shown in SEQ ID NO:12.

Optionally, the reovirus has a L1 genome segment having one or more nucleic acid modifications; a S4 genome segment having one or more nucleic acid modifications; a Ml genome segment having one or more nucleic acid modifications; and/or a M2 genome segment having one or more nucleic acid modifications, as described in U.S. Ser. No. 12/046,095, which is incorporated by reference herein in its entirety. By way of example, the one or more nucleic acid modifications in the L1 genome segment are a T at position 660, a G at position 817, an A at position 1687, a G at position 2283, an ATG at positions 2284-2286, a C at position 2794, a C at position 2905, a C at position 2953, an A at position 3153, or a G at position 3231, numbered relative to GenBank Accession No. M24734.1. Optionally, the L1 genome segment includes the sequence shown in SEQ ID NO:8. Further by way of example, the one or more nucleic acid modifications in the S4 genome segment is an A at position 74 and an A at position 624, numbered relative to GenBank Accession No. K02739. Optionally, the S4 genome segment includes the sequence shown in SEQ ID NO:4. Further by way of example, the nucleic acid modification in the M2 genome segment can be a C at position 248, numbered relative to GenBank Accession No. M20161.1. The M2 genome segment, for example, includes the sequence shown in SEQ ID NO:6. Also by way of example, the nucleic acid modification in the M1 genome segment is a T at position 1595, numbered relative to GenBank Accession No. AF461684.1. Optionally, the M1 genome segment includes the sequence shown in SEQ ID NO:5. A reovirus as described herein can include any modification or combination of modifications disclosed herein. Optionally, a reovirus as described herein includes genomic segments having the sequences shown in SEQ ID NOs:1-10 or the polypeptides shown in SEQ ID NOs:11, 12, and 16-21, and either or both SEQ ID NO:13 or 14. Optionally, a reovirus as disclosed herein is identified as IDAC Accession No. 190907-01.

Sindbis virus (SIN) can be used in the methods described herein. Sindbis virus is a member of the alphavirus genus of the togaviridae family. The Sindbis virus genome is a single-stranded RNA of 11703 nucleotides, capped at the 5′ terminus and poly-adenylated at the 3′ terminus. The genome consists of a 49S untranslated region (UT), nonstructural proteins nsP 1, nsP2, nsP3, and nsP4 followed by a promoter. The promoter is followed by a 26S UT, structural proteins C, E3, E2, 6K, and El and finally a 3′ UT and a poly-adenylated terminus. The genomic 49S RNA is of plus sense, is infectious, and serves as mRNA in the infected cell.

Sindbis vectors systemically and specifically infect/detect and kill metastasized tumors in vivo, leading to significant suppression of tumor growth and enhanced survival

(Hurtado et al., Rejuvenation Res. 9(1):36-44 (2006)). Sindbis virus infects mammalian cells using the Mr 67,000 laminin receptor, which is elevated in tumor versus normal cells. Tumor overexpression of the laminin receptor may explain the specificity and efficacy that Sindbis vectors demonstrate for tumor cells in vivo. Sindbis does not have to undergo genetic manipulation to target cancer cells or to be injected directly into tumors. Sindbis injected anywhere into a subject travels through the bloodstream to the target area (Tseng et al., Cancer Res. 64(18):6684-92 (2004). Sindbis can also be genetically engineered to carry one or more genes that suppress the immune response to the virus and/or genes that stimulate an immune response against the tumor such as, for example, antitumor cytokine genes such as interleukin-12 and interleukin-15 genes.

The oncolytic virus may be naturally occurring or modified. The virus may be chemically or biochemically pretreated (e.g., by treatment with a protease, such as chymotrypsin or trypsin) prior to administration to the neoplastic cells. Pretreatment with a protease removes the outer coat or capsid of the virus and may increase the infectivity of the virus. The virus may be coated in a liposome or micelle (Chandran and Nibert, J. of Virology 72(1):467-75 (1998)) to reduce or prevent an immune response from a mammal which has developed immunity to the virus. For example, the virion may be treated with chymotrypsin in the presence of micelle forming concentrations of alkyl sulfate detergents to generate a new infectious subvirion particle. The oncolytic virus may also be a reassortant virus or an ISVP.

The present methods include using any oncolytic virus according to the disclosure herein and knowledge available in the art. The oncolytic virus may be naturally occurring or modified. The oncolytic virus is naturally-occurring when it can be isolated from a source in nature and has not been intentionally modified by humans in the laboratory. For example, the oncolytic virus can be from a field source, that is, from a human who has been infected with the oncolytic virus.

The oncolytic virus may be a recombinant oncolytic virus. For example, the recombinant oncolytic virus results from the reassortment of genomic segments from two or more genetically distinct oncolytic viruses, also referred to herein as a reassortant. Reassortment of oncolytic virus genomic segments may occur following infection of a host organism with at least two genetically distinct oncolytic viruses. Recombinant viruses can also be generated in cell culture, for example, by co-infection of permissive host cells with genetically distinct oncolytic viruses. Optionally, the methods include the use of recombinant oncolytic virus resulting from reassortment of genome segments from two or more genetically distinct oncolytic viruses wherein at least one parental virus is genetically engineered, comprises one or more chemically synthesized genomic segment, has been treated with chemical or physical mutagens, or is itself the result of a recombination event. Optionally, the methods include the use of the recombinant oncolytic virus that has undergone recombination in the presence of chemical mutagens, including but not limited to dimethyl sulfate and ethidium bromide, or physical mutagens, including but not limited to ultraviolet light and other forms of radiation.

Optionally, the methods include the use of oncolytic viruses with mutations including (insertions, substitutions, deletions or duplications) in one or more genome segments. Such mutations can comprise additional genetic information as a result of recombination with a host cell genome, or that comprise synthetic genes such as, for example, genes encoding agents that suppress anti-viral immune responses.

Optionally, the oncolytic virus is a mutant oncolytic virus. For example, the oncolytic virus may be modified by incorporation of mutated coat proteins, such as for example, into the virion outer capsid. The mutant oncolytic virus is, optionally, a mutant reovirus. Mutant reoviruses as described herein can contain a mutation that reduces or essentially eliminates expression of a sigma3 polypeptide or that results in the absence of a functional sigma3 polypeptide as described in U.S. Ser. No. 12/124,522, which is incorporated by reference herein in its entirety. Optionally, the mutant reoviruses used in the provided methods are mutated as described in U.S. Serial No. 12/046,095, which is incorporated by reference herein in its entirety.

A mutation as referred to herein can be a substitution, insertion or deletion of one or more nucleotides. Point mutations include, for example, single nucleotide transitions (purine to purine or pyrimidine to pyrimidine) or transversions (purine to pyrimidine or vice versa) and single- or multiple-nucleotide deletions or insertions. A mutation in a nucleic acid can result in one or more conservative or non-conservative amino acid substitutions in the encoded polypeptide, which may result in conformational changes or loss or partial loss of function, a shift in the reading frame of translation (frame-shift) resulting in an entirely different polypeptide encoded from that point on, a premature stop codon resulting in a truncated polypeptide (truncation), or a mutation in a virus nucleic acid may not change the encoded polypeptide at all (silent or nonsense). See, for example, Johnson and Overington, 1993, J. Mol. Biol. 233:716-38; Henikoff and Henikoff, 1992, Proc. Natl. Acad. Sci. USA 89:10915-19; and U.S. Pat. No. 4,554,101, for disclosure on conservative and non-conservative amino acid substitutions.

Mutations can be generated in the nucleic acid of an oncolytic virus using any number of methods known in the art. For example, site directed mutagenesis can be used to modify a reovirus nucleic acid sequence. One of the most common methods of site-directed mutagenesis is oligonucleotide-directed mutagenesis. In oligonucleotide-directed mutagenesis, an oligonucleotide encoding the desired change(s) in sequence is annealed to one strand of the DNA of interest and serves as a primer for initiation of DNA synthesis. In this manner, the oligonucleotide containing the sequence change is incorporated into the newly synthesized strand. See, for example, Kunkel, 1985, Proc. Natl. Acad. Sci. USA 82:488; Kunkel et al., 1987, Meth. Enzymol. 154:367; Lewis and Thompson, 1990, Nucl. Acids Res. 18:3439; Bohnsack, 1996, Meth. Mol. Biol. 57:1; Deng and Nickoloff, 1992, Anal. Biochem. 200:81; and Shimada, 1996, Meth. Mol. Biol. 57:157. Other methods are used routinely in the art to modify the sequence of a protein or polypeptide. For example, nucleic acids containing a mutation can be generated using PCR or chemical synthesis, or polypeptides having the desired change in amino acid sequence can be chemically synthesized. See, for example, Bang and Kent, 2005, Proc. Natl. Acad. Sci. USA 102:5014-9 and references therein.

Viruses can be purified using standard methodology. See, for example, Schiff et al., “Orthoreoviruses and Their Replication,” Ch 52, in Fields Virology, Knipe and Howley, eds., 2006, Lippincott Williams and Wilkins; Smith et al., 1969, Virology 39(4):791-810; and U.S. Pat. Nos. 7,186,542; 7,049,127; 6,808,916; and 6,528,305, which are incorporated by reference herein in their entireties. As used herein, purified viruses refer to viruses that have been separated from cellular components that naturally accompany them. Typically, viruses are considered purified when they are at least 70% (e.g., at least 75%, 80%, 85%, 90%, 95%, or 99%) by dry weight, free from the proteins and other cellular components with which they are naturally associated.

Provided herein are pharmaceutical compositions comprising the oncolytic viruses. Also provided herein are pharmaceutical compositions comprising therapeutic agents, for example, the agents that decrease interstitial pressure and/or increase vascular permeability. Optionally, the pharmaceutical composition comprises the oncolytic virus and the agent that decreases interstitial pressure and/or increases vascular permeability. Optionally, the pharmaceutical composition comprises the oncolytic virus, the agent that decreases interstitial pressure and/or vascular permeability and the agent that inhibits pro-inflammatory cytokines. Thus, the provided pharmaceutical compositions can comprise one agent or more than one agent. For example, each of the oncolytic virus, the agent that decreases interstitial pressure and/or vascular permeability and the agent that inhibits pro-inflammatory cytokines can be contained within separate pharmaceutical compositions or the same composition. If the oncolytic virus and agents are contained within separate pharmaceutical compositions, the compositions can be administered concomitantly or sequentially.

The herein provided compositions are administered in vitro or in vivo in a pharmaceutically acceptable carrier. A pharmaceutically acceptable carrier can be a solid, semi-solid, or liquid material that can act as a vehicle, carrier or medium for the reovirus. Thus, compositions containing a reovirus and/or one or more of the provided agents can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Optionally, the compositions containing an oncolytic virus are suitable for infusion. For intravenous infusions, there are two types of fluids that are commonly used, crystalloids and colloids. Crystalloids are aqueous solutions of mineral salts or other water-soluble molecules. Colloids contain larger insoluble molecules, such as gelatin; blood itself is a colloid. The most commonly used crystalloid fluid is normal saline, a solution of sodium chloride at 0.9% concentration, which is close to the concentration in the blood (isotonic). Ringer's lactate or Ringer's acetate is another isotonic solution often used for large-volume fluid replacement. A solution of 5% dextrose in water, sometimes called D5W, is often used instead if the patient is at risk for having low blood sugar or high sodium.

Some examples of suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. A pharmaceutical composition additionally can include, without limitation, lubricating agents such as talc, magnesium stearate, and mineral oil; wetting agents; emulsifying and suspending agents; preserving agents such as methyl- and propylhydroxy-benzoates; sweetening agents; and flavoring agents. Pharmaceutical compositions can be formulated to provide quick, sustained or delayed release of a mutant reovirus after administration by employing procedures known in the art. In addition to the representative formulations described below, other suitable formulations for use in a pharmaceutical composition can be found in Remington: The Science and Practice of Pharmacy (21th ed.) ed. David B. Troy, Lippincott Williams & Wilkins, 2005. For preparing solid compositions such as tablets, a mutant reovirus can be mixed with a pharmaceutical carrier to form a solid composition. Optionally, tablets or pills can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid formulations that include a reovirus and/or agent for oral administration or for injection generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. These liquid or solid compositions may contain suitable pharmaceutically acceptable excipients as described herein. Such compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent or intermittent positive pressure breathing machine. Solution, suspension, or powder compositions may be administered, orally or nasally, from devices which deliver the formulation in an appropriate manner.

Another formulation that is optionally employed in the methods of the present disclosure includes transdermal delivery devices (e.g., patches). Such transdermal patches may be used to provide continuous or discontinuous infusion of the viruses and agents as described herein. The construction and use of transdermal patches for the delivery of pharmaceutical agents is well known in the art. See, for example, U.S. Pat. No. 5,023,252. Such patches can be constructed for continuous, pulsatile, or on-demand delivery of mutant reoviruses.

As described above, viruses and/or other agents can, if necessary, be coated in a liposome or micelle to reduce or prevent an immune response in a mammal that has developed immunity toward a virus or agent. Such compositions are referred to as immunoprotected viruses or agents. See, for example, U.S. Pat. Nos. 6,565,831 and 7,014,847.

In the provided methods, the oncolytic virus is administered, for example, systemically, in a manner so that it can ultimately contact the target tumor or tumor cells. The route by which the virus is administered, as well as the formulation, carrier or vehicle, depends on the location as well as the type of the target cells. A wide variety of administration routes can be employed. For example, for a solid tumor that is accessible, the virus can be administered by injection directly to the tumor. For a hematopoietic tumor, for example, the virus can be administered intravenously or intravascularly. For tumors that are not easily accessible within the body, such as metastases, the virus is administered in a manner such that it can be transported systemically through the body of the mammal and thereby reach the tumor (e.g., intravenously or intramuscularly). Alternatively, the virus can be administered directly to a single solid tumor, where it then is carried systemically through the body to metastases. The virus can also be administered subcutaneously, intraperitoneally, intrathecally or intraventricularly (e.g., for brain tumor), topically (e.g., for melanoma), orally (e.g., for oral or esophageal cancer), rectally (e.g., for colorectal cancer), vaginally (e.g., for cervical or vaginal cancer), nasally, by inhalation spray or by aerosol formulation (e.g., for lung cancer).

Optionally, the virus is administered continuously to a subject at least once per day or up to intermittently or continuously throughout the day on consecutive days, for a period of time. Thus, the virus is administered, for example, to subjects by means of intravenous administration in any pharmacologically acceptable solution, or as an infusion over a period of time. For example, the substance may be administered systemically by injection (e.g., IM or subcutaneously) or taken orally daily at least once per day, or administered by infusion in a manner that results in the daily delivery into the tissue or blood stream of the subject. When the virus is administered by infusion over a period of time, the period of time is, for example, 1, 2, 3, 4, 5, 6, 7, 8, 9, 10, 12, or 24 hours, or any time between 1 and 24 hours, inclusive, or more. Optionally, the period of time is 5, 15, 30, 60, 90, 120, 150 or 180 minutes, or any time between 5 and 180 minutes, inclusive, or more. Thus, for example, the virus is administered by infusion for 60 minutes. Administrations can be repeated daily for 2, 3, 4, 5, 6, 7, 8, 9, 10, 14, 21, 28 days or any number of days between 2 and 28 days, inclusive, or longer.

The agents that decrease interstitial pressure and/or vascular permeability or other therapeutic agents (i.e., the agents that inhibit pro-inflammatory cytokines) of the provided methods are also administered via a wide variety of administration routes. Thus, the agents are administered via any of several routes of administration, including, topically, orally, parenterally, intravenously, intraperitoneally, intramuscularly, subcutaneously, intracavity, transdermally, intrahepatically, intracranially, nebulization/inhalation, or by instillation via bronchoscopy. Optionally, the therapeutic agents are administered continuously in the manner set forth in the description above with respect to oncolytic viruses. Thus, for example, the agent is administered to subjects by means of intravenous administration in any pharmacologically acceptable solution, or as an infusion over a period of time. Optionally, the agents are administered locally at or near the site of the tumor. Alternatively, the agents are administered systemically. The agents that decrease interstitial pressure and/or vascular permeability are administered in an amount that is sufficient (i.e., an effective amount) to decrease interstitial pressure and/or increase vascular permeability. Agents that inhibit pro-inflammatory cytokines are administered in an amount sufficient (i.e., an effective amount) to inhibit one or more pro-inflammatory cytokines. By way of example, effective amounts of taxanes include from about 40-300 mg/m² of tumor volume; or any amount in between 40 and 300 mg/m², inclusive. Thus, effective amounts of taxanes include 130-225 mg/m². By way of another example, effective amounts of platinum compounds include from about 5-1000 mg/m², or any amount in between 5 and 1000 mg/m², inclusive. Thus, for example effective amounts of cisplatin include from about 175-200 mg/m² and effective mounts for carboplatin include from about 200-600 mg/m². Effective amounts of other agents range from 0.001-10,000 mg/kg body weight or any amount in between 0.001 and 10,000 mg/kg body weight, inclusive. Optionally, effective amounts of platinum compounds include approximately 2 to 7 mg/mL minute (AUC) as calculated by the Calvert formula. Optionally, effective amounts of platinum compounds include approximately 5 or 6 mg/mL minute (AUC) as calculated by the Calvert formula. Optionally, the platinum compounds are administered as an intravenous infusion over a period of 30 minutes. The viruses as disclosed herein are administered in an amount that is sufficient (i.e., an effective amount) to treat the proliferative disorder. A proliferative disorder is treated when administration of a virus to proliferating cells affects lysis (e.g., oncolysis) of the affected cells, resulting in a reduction in the number of abnormally, proliferating cells, a reduction in the size of a neoplasm, and/or a reduction in or elimination of symptoms (e.g., pain) associated with the proliferating disorder. As used herein, the term oncolysis means at least 10% of the proliferating cells are lysed (e.g., at least about 20%, 30%, 40%, 50%, or 75% of the cells are lysed). The percentage of lysis can be determined, for example, by measuring the reduction in the size of a neoplasm or in the number of proliferating cells in a mammal, or by measuring the amount of lysis of cells in vitro (e.g., from a biopsy of the proliferating cells). An effective amount of a virus will be determined on an individual basis and may be based, at least in part, on the particular virus used; the individual's size, age, gender; and the size and other characteristics of the abnormally, proliferating cells. For example, for treatment of a human, approximately 10³ to 10¹² plaque forming units (PFU) of a virus are used, depending on the type, size and number of proliferating cells or neoplasms present. The effective amount can be, for example, from about 1.0 PFU/kg body weight to about 10¹⁵ PFU/kg body weight (e.g., from about 10² PFU/kg body weight to about 10¹³ PFU/kg body weight). Optionally, the effective amount is about 1×10⁸ to about 1×10¹² TCID₅₀. Optionally, the effective amount is about 1×10¹⁰ TCID₅₀.

By way of example, 175 mg/m² of the agent that decreases interstitial pressure and/or increases vascular permeability, such as paclitaxel, is administered to the subject and 3×10¹⁰ TCID₅₀ or 1×10¹⁰ TCID₅₀ of a reovirus is administered to the subject. Optionally, 200 mg/m² of the agent that decreases interstitial pressure and/or increases vascular permeability, such as paclitaxel, is administered to the subject and 3×10¹⁰ TCID₅₀ or 1×10¹⁰ TCID₅₀ of a reovirus is administered to the subject. Optionally, the agent that decreases interstitial pressure and/or increases vascular permeability is administered as a three hour intravenous infusion. Optionally, the reovirus is administered as a one hour intravenous infusion.

By way of another example, 175 mg/m² of the agent that decreases interstitial pressure and/or increases vascular permeability, such as paclitaxel, is administered to the subject; 5 mg/ml minute (AUC as calculated by the Calvert formula) of an agent that inhibits pro-inflammatory cytokines, such as carboplatin, is administered to the subject; and 3×10¹⁰ TCID₅₀ or ×10¹⁰ TCID₅₀ of a reovirus is administered to the subject. Optionally, 200 mg/m² of the agent that decreases interstitial pressure and/or increases vascular permeability, such as paclitaxel, is administered to the subject; 6 mg/ml minute of an agent that inhibits pro-inflammatory cytokines is administered to the subject; and 3×10¹⁰ TCID₅₀ or 1×10¹⁰ TCID₅₀ of a reovirus is administered to the subject. Optionally, the agent that decreases interstitial pressure and/or increases vascular permeability is administered as a three hour intravenous infusion. Optionally, the agent that inhibits pro-inflammatory cytokines is administered as a thirty minute intravenous infusion. Optionally, the reovirus is administered as a one hour intravenous infusion.

Optimal dosages of viruses and therapeutic agents and compositions comprising viruses and agents depend on a variety of factors. The exact amount required will vary from subject to subject, depending on the species, age, weight and general condition of the subject, the severity of the disease being treated, the particular virus or vector used and its mode of administration. Thus, it is not possible to specify an exact amount for every composition. However, an appropriate amount can be determined by one of ordinary skill in the art using only routine experimentation given the guidance provided herein.

Effective dosages and schedules for administering the compositions may be determined empirically. For example, animal models for a variety of proliferative disorders can be obtained from the Jackson Laboratory, 600 Main Street, Bar Harbor, Me. 04609 USA. Both direct (e.g., histology of tumors) and functional measurements (e.g., survival of a subject or size of a tumor) can be used to monitor response to therapies. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy.

The dosage ranges for the administration of compositions are those large enough to produce the desired effect in which the symptoms of the disease are affected. The dosage should not be so large as to cause adverse side effects, such as unwanted cross-reactions and anaphylactic reactions. The dosage can be adjusted by the individual physician in the event of any counterindications.

Dosages vary and are administered in one or more dose administrations daily, for one or several days. The provided viruses and therapeutic agents are administered in a single dose or in multiple doses (e.g., two, three, four, six, or more doses). For example, where the administration is by infusion, the infusion can be a single sustained dose or can be delivered by multiple infusions. Treatment may last from several days to several months or until diminution of the disease is achieved.

Combinations of the provided viruses and therapeutic agents are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents. By way of example, the agent that decreases interstitial pressure is administered prior to or at the same time as the oncolytic virus. By way of another example, the agent that decreases interstitial pressure is administered first or second, the agent that inhibits a pro-inflammatory cytokine is administered first or second and the oncolytic virus is administered third. Optionally, the agent that decreases interstitial pressure is administered first, and the agent that inhibits a pro-inflammatory cytokine is administered at the same time as the oncolytic virus. When one compound is administered prior to another compound, the first compound is administered minutes, hours, days, or weeks prior to administration of the second compound. For example, the first compound can be administered at 1, 2, 3, 4, 5, 6, 7, 8, 9, 10, 12, 24, 36, 48, 60, or 72 hours, or any time between 1 and 72 hours, inclusive, prior to administration of a second compound. Optionally, the first compound is administered more than 72 hours prior to the second compound. By way of another example, the first compound can be administered at 1, 5, 15, 30, 60, 90, 120, 150 or 180 minutes, or any time between 1 and 180 minutes, inclusive, prior to administration of a second compound. Optionally, the first compound is administered at 1, 2, 3, 4, 5, 6, 7, 14, 21, or 28 days, or any amount in between 1 and 28, inclusive, days prior to administration of the second compound. Optionally, the first compound is administered more than 28 days prior to the second compound. For example, the agent(s) that decreases interstitial pressure and/or increases vascular permeability is administered from about 1 to 8 hours prior to administration of the oncolytic virus. By way of another example, the agent(s) that decreases interstitial pressure and/or increases vascular permeability is administered first at a time of four, six, eight or ten hours prior to administration of the oncolytic virus, the agent that inhibits pro-inflammatory cytokines is administered second at a time of one hour prior to administration of the oncolytic virus and the oncolytic virus is administered third (i.e., one hour after administration of the agent that inhibits pro-inflammatory cytokines).

Oncolytic viruses or a pharmaceutical composition comprising such viruses are optionally packaged into a kit. The kit also includes one or more agents or pharmaceutical compositions comprising such agents that decrease interstitial pressure and/or increase vascular permeability. The kit, optionally, also includes one or more agents that inhibit a pro-inflammatory cytokine, one or more chemotherapeutic agents, one or more immunosuppressive agents, and/or one or more anti-anti-virus antibodies. A pharmaceutical composition can be formulated in a unit dosage form. The term “unit dosage forms” refers to physically discrete units suitable as unitary dosages for human subjects and other mammals, each unit containing a predetermined quantity of a mutant reovirus calculated to produce the desired therapeutic effect in association with a suitable pharmaceutically acceptable carrier.

The provided methods may be combined with other tumor therapies such as chemotherapy, radiotherapy, surgery, hormone therapy and/or immunotherapy. Thus, the oncolytic virus may be administered in conjunction with surgery or removal of the neoplasm. Therefore, provided herewith are methods for the treatment of a solid neoplasm comprising surgical removal of the neoplasm and administration of an oncolytic virus at or near to the site of the neoplasm.

The compositions in the provided methods are, optionally, administered in conjunction with or in addition to known anticancer compounds or chemotherapeutic agents. Chemotherapeutic agents are compounds which may inhibit the growth of tumors. Such agents, include, but are not limited to 5-fluorouracil; mitomycin C; methotrexate; hydroxyurea; cyclophosphamide; dacarbazine; mitoxantrone; anthracyclins (epirubicin and doxurubicin); antibodies to receptors, such as herceptin; etoposide; pregnasome; hormone therapies such as tamoxifen and anti-estrogens; interferons; aromatase inhibitors; progestational agents; and LHRH analogs.

As used herein, the term proliferative disorder refers to any cellular disorder in which the cells proliferate more rapidly than normal tissue growth. A proliferative disorder includes, but is not limited to, neoplasms, which are also referred to as tumors. A neoplasm can include, but is not limited to, pancreatic cancer, breast cancer, brain cancer (e.g., glioblastoma), lung cancer, prostate cancer, colorectal cancer, thyroid cancer, renal cancer, adrenal cancer, liver cancer, neurofibromatosis 1, and leukemia. A neoplasm can be a solid neoplasm (e.g., sarcoma or carcinoma) or a cancerous growth affecting the hematopoietic system (e.g., lymphoma or leukemia). Other proliferative disorders include, but are not limited to neurofibromatosis.

Generally, in proliferating disorders for which oncolytic virus is used as a treatment, one or more of the proliferating cells associated with the disorder may have a mutation in which the ras gene (or an element of the ras signaling pathway) is activated, either directly (e.g., by an activating mutation in ras) or indirectly (e.g., by activation of an upstream or downstream element in the ras pathway). Activation of an upstream element in the ras pathway includes, for example, transformation with epidermal growth factor receptor (EGFR) or Sos. See, for example, Wiessmuller and Wittinghofer, 1994, Cellular Signaling 6(3):247-267; and Barbacid, 1987, Ann. Rev. Biochem. 56, 779-827. Activation of a downstream element in the ras pathway includes, for example, mutation within B-Raf. See, for example, Brose et al., 2002, Cancer Res. 62:6997-7000. A proliferative disorder that results, at least in part, by the activation of ras, an upstream element of ras, or an element in the ras signaling pathway is referred to herein as a ras-mediated proliferative disorder. In addition, the oncolytic virus is useful for treating proliferative disorders caused by mutations or dysregulation of PKR. See, for example, Strong et al., 1998, EMBO J. 17:3351-62.

As used herein the terms treatment, treat, treating or ameliorating refers to a method of reducing the effects of a disease or condition or symptom of the disease or condition. Thus in the disclosed method, treatment can refer to a 10%, 20%, 30%, 40%, 50%, 60%, 70%, 80%, 90% or 100% reduction or amelioration in the severity of an established disease or condition or symptom of the disease or condition. For example, the method for treating cancer is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the term subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, whether male or female, are intended to be covered. As used herein, patient or subject may be used interchangeably and can refer to a subject with a disease or disorder. The term patient or subject includes human and veterinary subjects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of the inhibitor, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

### EXAMPLE

### Example 1

### Reovirus, Paclitaxel and Carboplatin Protocols for Humans

This is a study design of reovirus given intravenously with paclitaxel and carboplatin every 3 weeks.

Paclitaxel is administered as a 3 hour intravenous infusion at a dose of 175 mg/m² or 200 mg/m². Carboplatin is then administered as a 30 minute intravenous infusion at a dose calculated by the Calvert formula (AUC 5 mg/mL minute or 6 mg/mL minute with GFR measured by 51Cr EDTA). After paclitaxel and carboplatin administration, reovirus is then administered as a 1 hour intravenous infusion at a dose of 1×10¹⁰ or 3×10¹⁰ TCIDso

On days 2 through 5, only reovirus will be administered, using the same dose and method as used on Day 1.

TABLE 2
Dosing Methods

| | | Carboplatin | |
|---|---|---|---|
| | | Dose | |
| | Paclitaxel | AUC mg/mL | Reovirus dose |
| | Dose (mg/m²) | min | (TCID₅₀) |
| | Day 1 only | Day 1 only | Days 1-5 |
| Method 1 | 175 | 5 | 1 × 10¹⁰ |
| Method 2 | 175 | 5 | 3 × 10¹⁰ |
| Method 3 | 200 | 6 | 1 × 10¹⁰ |
| Method 4 | 200 | 6 | 3 × 10¹⁰ |

### Example 2

### Reovirus and mTOR Inhibitors

Using a constant ratio combination design and combination index method based on the Chou and Talalay median-effect principle (Chou and Talalay, Trends Pharmacol. Sci. 4:450-454 (1983)), the effect of reovirus combined with rapamycin on B16.F10 cells was assessed.

Cells (5×10³/well) were seeded in 96 well plates and allowed to adhere overnight. Culture medium was replaced with doubling dilutions of rapamycin and/or reovirus, corresponding to 2, 1, 0.5 and 0.25 times the previously determined ED50, diluted in fresh culture medium and incubation continued for 48 h. At this time, medium was removed and percentage cell survival compared to untreated cells was determined using the MTS assay. Data were analyzed using the CalcuSyn program.

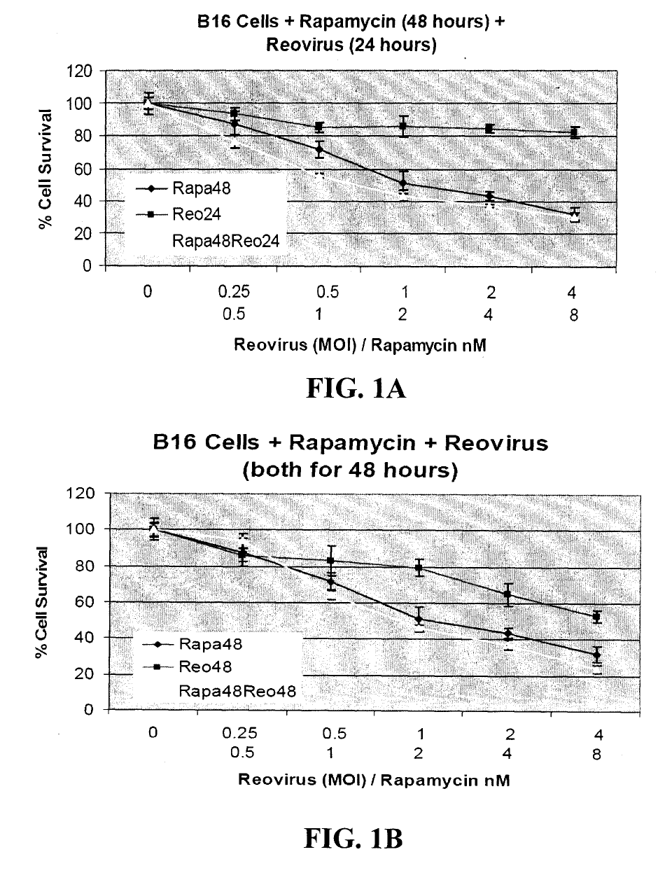

The effect of sequencing was assessed by adding the rapamycin 24 hours before or after the reovirus. Of note, at 24 h little if any cell death was seen with reovirus. The interaction was antagonistic (combination index value (CIV) of more than one) if the rapamycin preceded or was given concomitantly with reovirus (FIGS. 1a and 1b, respectively). A synergistic interaction (CIV of less than one) was observed between reovirus and rapamycin only when the rapamycin was given after the reovirus (FIG. 1c).

In the in vivo setting, combined reovirus and rapamycin therapy reduced the growth of subcutaneously implanted tumors and prolonged the median survival time of mice. B16.F10 tumors were seeded subcutaneously in C57B1/6 mice and treated with intratumoral reovirus T3D 5×10⁸ TCID50 on day 1 and 4, and intraperitoneal rapamycin 5 mg/kg on day 1, 4, 8 and 12 either alone or in combination, or with control treatment (intratumoural PBS, intraperitoneal PBS).

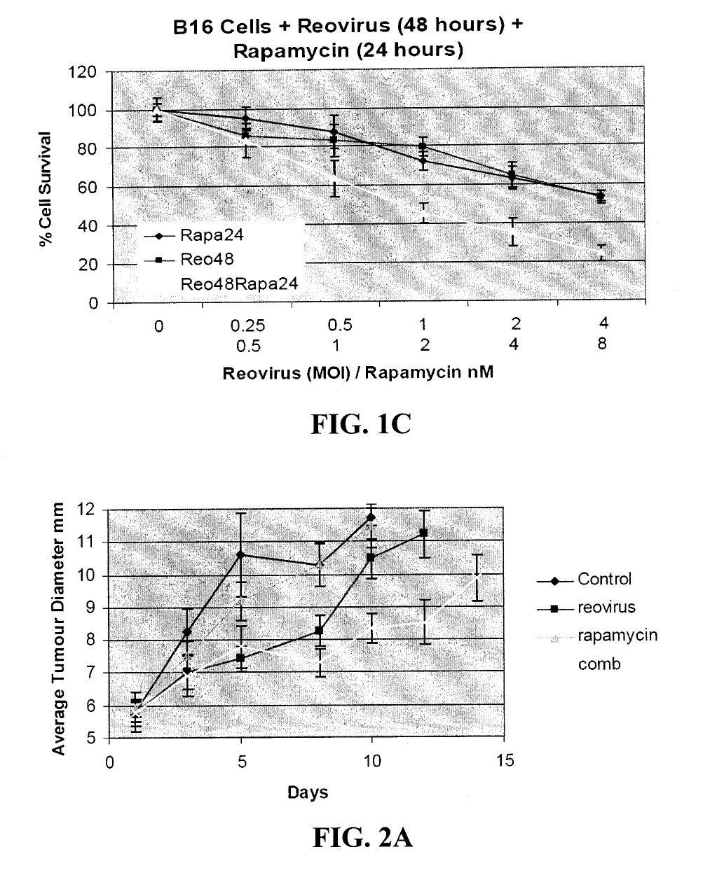

The diameter of each tumor was measured and an average calculated for each group. Combined reovirus T3D/rapamycin treatment resulted in markedly reduced tumor growth compared to single agent treatments or control treatment (FIG. 2A).

Survival was plotted as a Kaplan-Meier curve. Median survival time for control treated mice was 7 days. There was no improvement in median survival with rapamycin alone. Reovirus alone prolonged median survival time to 9 days. Combined therapy increased survival time to >15 days (Logrank test p=0.0216) (FIG. 2B).

### Example 3

### Reovirus, Cyclophosphamide (CPA) and IL-2

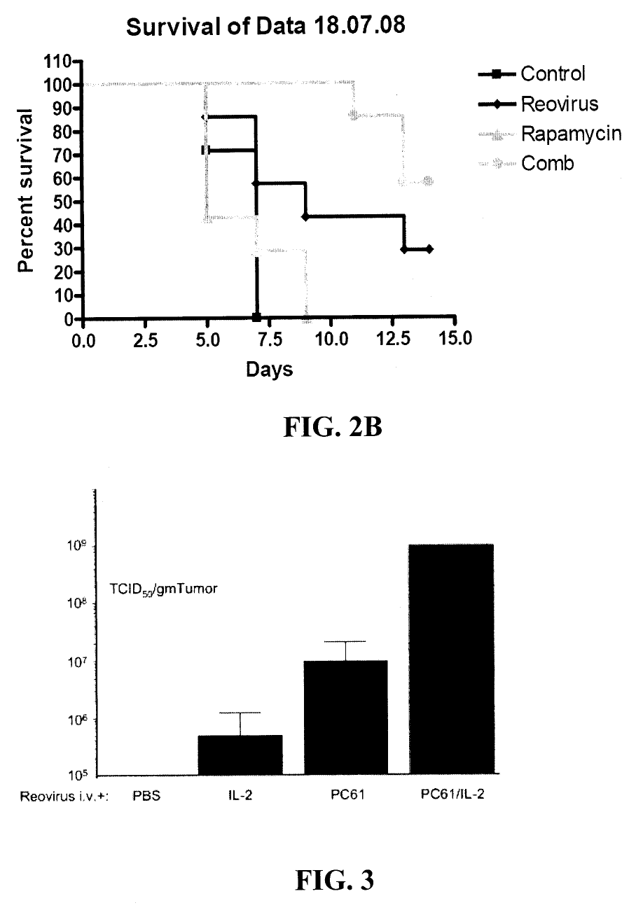

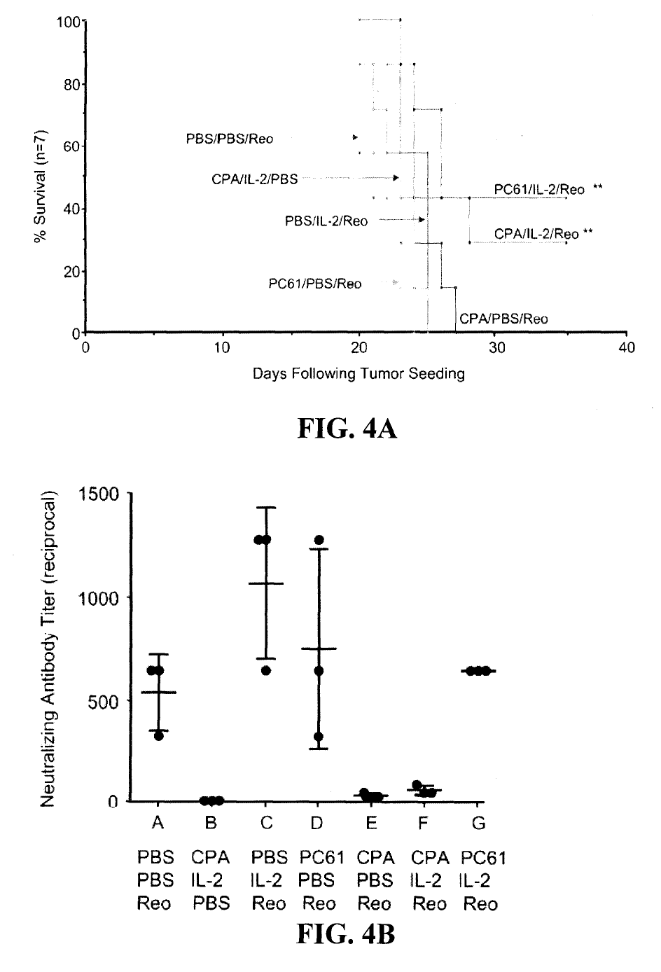

Preconditioning of C57B1/6 mice with Treg depletion (PC-61) and/or IL-2 enhanced the localization of intravenously delivered reovirus to subcutaneous, established B16 tumors (FIG. 3). However, the high dose of reovirus (3.75×10⁹ TCID₅₀) used in this experiment resulted in toxicities. Therefore, the therapeutic efficacy of PC-61 or CPA (which mimics the effects of PC-61)+IL-2+reovirus was tested wherein the viral dose of reovirus was reduced to 1×10⁸ TCID50 per injection. Under these conditions, equivalent therapy of subcutaneous B 16 tumors was observed using either PC-61+IL-2 or CPA+IL-2 at levels that were significantly better than any of the control treatments (P<0.01; FIG. 4A). None of the mice treated with the preconditioning regimens and intravenous reovirus developed toxicities. Despite the lack of observable toxicity, reovirus was, however, recovered from both the lungs and the hearts of mice treated with CPA+IL-2+reovirus. This is in contrast to mice treated with PC-61+IL-2+reovirus where virus was recovered only from the lungs and not from the hearts. Therefore, preconditioning with CPA+IL-2 enhanced the therapy produced by systemic delivery of intravenously delivered reovirus to a level indistinguishable from that induced by PC-61+IL-2.

Previously, it was shown that a higher dose of CPA (150 mg/kg) can modulate levels of NAb against reovirus to allow for repeat administration of the virus (Qiao et al., Clin. Cancer Research 14:259-69 (2008)). Therefore, although NAb to reovirus has not been shown to have any inhibitory role in the therapeutic effects seen in the virus-naive C57B1/6 mice in FIG. 4A, their serum was tested for levels of NAb. As expected, serum from mice treated with reovirus alone contained high levels of neutralizing activity against reovirus (FIG. 4B). Pretreatment with either IL-2 or PC-61 showed a trend toward increasing the level of neutralizing activity in the serum, although these values were very variable. Pretreatment with CPA before reovirus administration reduced this neutralizing activity significantly (P <0.01), which was maintained with the combination of CPA+IL-2 (FIG. 4B). Combination of Treg depletion by PC-61+IL-2 maintained levels of neutralization at those observed in mice treated with reovirus alone (FIG. 4B). Therefore, use of CPA in combination with IL-2+reovirus not only enhances antitumor therapy (FIG. 4A) but also modulates levels of anti-reovirus antibody.

In summary, these data show that PC-61+IL-2 enhanced intratumoral localization of systemically delivered reovirus by 2 to 3 logs compared with mice treated with PBS/reovirus alone. This is due to IL-2-induced vascular leakage at the tumor site, which increased the ability of systemically delivered virus to localize into established tumors. Further, the data show that CPA-mediated Treg modification, with IL-2 and reovirus, is therapeutic against established tumors.

1. A method for treating a proliferative disorder in a subject, comprising the steps of:
(a) decreasing interstitial pressure in the subject; and
(b) administering to the subject one or more oncolytic viruses.

2. The method of claim 1, wherein approximately 10³ to 10¹² plaque forming units (PFU) of the oncolytic virus is administered to the subject.

3. The method of claim 2, wherein approximately 10⁸ to 10¹² plaque forming units (PFU) of the oncolytic virus is administered to the subject.

4. The method of claim 1, wherein approximately 10⁸ to 10¹² TCID₅₀ of the oncolytic virus is administered to the subject.

5. The method of claim 1, wherein step (a) is carried out by administering to the subject an agent that decreases interstitial pressure.

6. The method of claim 5, wherein approximately 5 to 1000 mg/m² of the agent that decreases interstitial pressure is administered to the subject.

7. The method of claim 5, wherein approximately 0.001-10,000 mg/kg body weight of the agent that decreases interstitial pressure is administered to the subject.

8. The method of claim 5, wherein the agent that decreases interstitial pressure increases vascular permeability.

9. The method of claim 5, wherein the agent that decreases interstitial pressure is a taxane.

10. The method of claim 6, wherein the taxane is selected from the group consisting of larotaxel, paclitaxel and docetaxel.

11. The method of claim 9, wherein approximately 40-300 mg/m² of the taxane is administered to the subject.

12. The method of claim 9, wherein approximately 130-225 mg/m² of the taxane is administered to the subject.

13. The method of claim 10, wherein approximately 175-200 mg/m² of the paclitaxel is administered to the subject.

14. The method of claim 5, wherein the agent is selected from the group consisting of interleukin-1 (IL-I), interferon-K (IFN-K), substance P, a proteinase inhibitor, vascular endothelial growth factor (VEGF), nitroglycerine, serotonin, a plasma kinin, platelet-activating factor (PAF), prostaglandin El (PGE1), histamine, imatinib, zona occludens toxin (ZOT), interleukin-2, a nitric oxide inhibitor, and a human growth factor receptor tyrosine kinase inhibitor.

15. The method of claim 14, wherein the proteinase inhibitor is N-alpha-tosyl-L-lysyl-chloromethyl-ketone (TLCK), tosyl phenylalanyl chloromethyl ketone (TPCK) or leupeptin.

16. The method of claim 14, wherein the plasma kinin is bradykinin.

17. The method of claim 14, wherein the nitric oxide inhibitor is L-N-monomethyl arginine (L-NMMA) or L-N-nitro-arginine methyl ester (L-NAME).

18. The method of claim 1, wherein step (a) is carried out by administering to the subject a low calcium ion concentration fluid.

19. The method of claim 18, wherein the fluid comprises a calcium ion concentration of 50 Tmol/L to 200 Tmol/L.

20. The method of claim 1, wherein step (a) is carried out by removing excess interstitial fluid at or near the site of the proliferative disorder.

21. The method of claim 20, wherein the excess interstitial fluid is removed by artificial lymphatic system (ALS).

22. The method of claim 1, wherein step (a) is carried out by administering to the subject a permeabilizing photodynamic therapeutic agent.

23. The method of claim 1, wherein step (a) is carried out at the same time, before or after step (b).

24. The method of claim 5, wherein the agent that decreases interstitial pressure is administered before the oncolytic virus.

25. The method of claim 24, wherein the agent is administered from 1 to 12 hours before the oncolytic virus.

26. The method of claim 1, wherein the virus is administered in multiple doses.

27. The method of claim 5, wherein the agent that decreases interstitial pressure is administered in multiple doses.

28. The method of claim 5, further comprising the step of administering to the subject an agent that inhibits a pro-inflammatory cytokine.

29. The method of claim 28, wherein the agent inhibits a pro-inflammatory cytokine but does not inhibit or minimally inhibits production of NARA.

30. The method of claim 28, wherein the agent that inhibits a pro-inflammatory cytokine is a platinum compound.

31. The method of claim 30, wherein the platinum compound is selected from the group consisting of cisplatin, carboplatin and oxaliplatin.

32. The method of claim 30, wherein approximately 5-1000 mg/m² of the platinum compound is administered to the subject.

33. The method of claim 31, wherein 2 to 7 mg/mL minute (AUC) of the carboplatin is administered to the subject.

34. The method of claim 31, wherein 5 or 6 mg/mL minute (AUC) of the carboplatin is administered to the subject.

35. The method of claim 28, wherein the agent that decreases interstitial pressure is paclitaxel, the agent that inhibits a pro-inflammatory cytokine is carboplatin and the oncolytic virus is a reovirus.

36. The method of claim 28, wherein the agent that decreases interstitial pressure is administered first at a time of four hours prior to administration of the oncolytic virus and wherein the agent that inhibits a pro-inflammatory cytokine is administered second at a time of one hour prior to administration of the oncolytic virus.

37. The method of claim 1 , wherein the virus has one or more mutations or deletions so as not to inhibit the double-stranded RNA activated protein kinase (PKR).

38. The method of claim 1, wherein the oncolytic virus is selected from the group consisting of reovirus, sindbis virus, Delta24, vesicular stomatitis virus (VSV), Newcastle disease virus (NDV), vaccinia virus, encephalitis virus, herpes zoster virus, hepatitis virus, influenza virus, varicella virus, and measles virus.

39. The method of claim 38, wherein the reovirus is a mammalian reovirus.

40. The method of claim 38, wherein the reovirus is a human reovirus.

41. The method of claim 40, wherein the human reovirus is selected from the group consisting of serotype 1 reovirus, serotype 2 reovirus and serotype 3 reovirus.

42. The method of claim 40, wherein the human reovirus is serotype 3 reovirus.

43. The method of claim 38, wherein the reovirus has IDAC Accession No. 190907-01.
